(19) United States
(12) Patent Application Publication
(10) Pub. No.: US 2026/0199593 A1
(43) Pub. Date: Jul. 16, 2026

(54) IMPLANTABLE INFUSION PUMP AND RELATED METHODS

(71) Applicant: Gordy Medical, LLC, Houston, TX (US)

(72) Inventors: Daniel Drew GALYON, Ottsville, PA (US); Richard Arthur HOWES, JR., Franklin, TN (US); Stephen KEATING, Pompton Lakes, NJ (US); Ernest Ketterer, Kinnelon, NJ (US); Michael TURI, Hackettstown, NJ (US); Jeffrey Daniel TRIOLO, New York, NY (US); Andreas GUBLER, New York, NY (US)

(73) Assignee: Gordy Medical, LLC, Houston, TX (US)

(21) Appl. No.: 19/444,826

(22) Filed: Jan. 9, 2026

Related U.S. Application Data
(60) Provisional application No. 63/745,406, filed on Jan. 15, 2025.
(60) Provisional application No. 63/864,802, filed on Aug. 15, 2025.

(51) Int. Cl. A61M 5/142; A61M 5/145; A61M 39/00; A61M 39/22

(52) U.S. Cl. CPC A61M 5/14276; A61M 5/14586; A61M 39/22; A61M 2005/14264; A61M 2039/0009; A61M 2202/0007; A61M 2202/0468; A61M 2205/0216; A61M 2205/0294; A61M 2205/04; A61M 2205/3303; A61M 2205/3327; A61M 2205/3344; A61M 2205/3576; A61M 2205/502; A61M 2205/8206; A61M 2210/0687; A61M 2230/005; A61M 2230/62

(57) ABSTRACT

In some aspects the present systems can include an implantable medical device (IMD) configured to be disposed within the patient. The IMD may include a housing defining a reservoir configured to receive and store an active substance, a chamber configured to receive the active substance from the reservoir, a catheter port configured to receive the active substance from the reservoir, and a linear piezoelectric actuator configured to initiate delivery of the active substance from the chamber to the catheter port. In some configurations, the system can include external interface control devices, external medical accessories, or both.

This application claims priority of U.S. Provisional Patent Application No. 63/745,406, filed Jan. 15, 2025 and U.S. Provisional Ser. No. 63/864,802, filed Aug. 15, 2025, which are hereby incorporated by reference in their entirety.

### TECHNICAL FIELD

The present application relates generally to implantable medical devices (IMDs) and, more specifically, to convection enhanced delivery systems and methods for delivering therapeutics to the brain of the patient and monitoring the pressure of the brain.

### BACKGROUND

Medical pumps can be used to treat a variety of physiological, psychological, and emotional conditions. For some medical conditions, medical pumps can restore an individual to a more healthful condition and a fuller life. For example, medical pumps may be used for chronic delivery of therapeutic agents, such as drugs. As one specific example, a medical pump may be used to deliver insulin to a diabetic patient. Other examples include delivery of pain relief medication, e.g., to the intrathecal or epidural space of a patient, to alleviate chronic pain. However, as stated in U.S. Pat. No. 11,331,424, it is understood that medical pumps do not lend themselves to all medical applications and more particularly to applications for delivery of medication to the brain. For example, in current intrathecal pumps, medication is meant to be localized in the spine and such pumps are unable to effectively deliver medication to the brain without significant risks. U.S. Patent Pub. 2022/0134076 describes several challenges that prevented previous attempts of drug delivery to deep brain structures within the brain.

Current methods for direct drug delivery to the brain utilize an Ommaya reservoir that can be placed under the scalp of the patient. Pharmaceutical agents can then be intermittently injected into the Ommaya reservoir and delivered to the brain via the catheter. While current devices are capable of delivering a pharmaceutical agent to a patient's brain, these methods are often limited and do not offer functionality that can greatly benefit a patient in their treatment.

### SUMMARY

The present disclosure provides apparatuses, systems, and methods for delivery of therapeutic or pharmaceutical agents via an implantable medical device that can allow for increased functionality and use of implantable systems. In some aspects, the systems and methods described herein can include or be configured for convection enhanced delivery (CED) (e.g., continuous or intermittent CED). Configurations of the present pump system can utilize an implanted medical device (IMD) having a catheter that can connect the IMD to a target area (e.g., ventricle, vein, artery, central nervous system, peripheral nervous system, tissue, or the like) thereby allowing fluid communication between the IMD and the target area. In some configurations, the target area may be a ventricle of the brain or, alternatively, an area of resection (e.g., portion of organ or tissue where a tumor has been removed). For example, the IMD can be disposed within an abdomen of the patient and connected with a catheter that includes a proximal end coupled to the IMD (e.g., in the abdomen) and a distal end that is disposed within a treatment area of the patient, such as a vein, artery, ventricle, organ, or other area. A variety of sensors (e.g., a pressure sensor, fluid sensors, or the like) can be connected to the catheter to allow for continual monitoring of a patient's intracranial pressure (ICP). The IMD can also include other sensors (e.g., accelerometer, electrical sensors) to assist with patient monitoring. These measurements can prove vital and extremely useful in facilitating the delivery of pharmaceutical agents to the treatment area. For example, where the treatment area is a ventricle of the brain, if the ICP is greater than a certain threshold, continual delivery of the agent to the ventricle can be stopped to mitigate harm to the patient. Additionally, or alternatively, ICP waveforms can be utilized to determine information about a patient that is not available using conventional methods of drug delivery (e.g., through CSF). In some aspects, an orientation of the IMD is utilized to determine a position of the patient. For example, based on a determination that the IMD is positioned in a first orientation, the controller can determine that the patient is in an upright position. In configurations in which the patient orientation is relevant, the controller may be configured to alert the patient to assume the correct position. In these ways and others, the present system can allow for enhanced and safer treatment of a patient.

In some aspects, the IMD can transmit information associated with the patient to an external device (e.g., tablet, computer, medical alert device, or the like). In some configurations, the external device can be associated for use by the patient or, alternatively, by another (e.g., a medical professional). For example, a user device can issue alerts to the patient and provide instruction to provide easy use of the system to the patient. Alternatively, a professional device can provide updates on the patient status, store patient data, and/or enable actions that facilitate the medical treatment of the patient (e.g., change dosage of a medication). The system, user device, and professional device can communicate with each other via a variety of communication protocols, thereby allowing for consistent monitoring of the patient and IMD status. Some external devices can include a display or other indication device (e.g., light, speaker, vibrator, or the like) configured to switch between states based on the information from the IMD. For example, the external device may be configured such that, based on the external device receiving information associated with the ICP waveform, the external device can display a predetermined user interface state (e.g., display of ICP wave information, high pressure alert, or the like). Additionally, or alternatively, the controller may be configured such that, based on a determination that the IMD is positioned in a first orientation that is associated with the patient being in an upright position, the controller can transmit a signal to the external device to cause the device to display a respective user interface state (e.g., orientation alert).

In some aspects of the present system, the external device can include one or more sensors that can, but need not be, in communication with the IMD. In a non-limiting example, the sensor may include a pressure sensor configured to determine an ambient pressure in an environment of the patient. In some such examples, the controller (of the IMD, external device, or both) can be configured to adjust the information associated with the ICP waveform based on the ambient pressure or provide a gauge pressure measurement. In some aspects, the system can include an intermediary device that is configured to communicate with the IMD via a first protocol and that is configured to communicate with the external device via a second protocol.

The IMD of the present system can contain a variety of elements, including (but not limited to): a reservoir, an accumulator, an actuator, valves, springs, and/or ports to allow for fluid delivery into the system and fluid delivery to the catheter. In some configurations, the IMD can include a housing defining a reservoir configured to receive and store an active substance, a chamber configured to receive the active substance from the reservoir, and/or a catheter port configured to receive the active substance from the reservoir. In some aspects, the IMD can include an actuator disposed within the housing and configured to move between a first state and a second state to initiate delivery of the active substance from the chamber to the catheter port. The actuator can be configured to move between the first and second states in a linear manner and, in some configurations, the actuator can be a piezoelectric driven actuator.

In some of the present systems, the actuator can directly or indirectly increase a pressure within an accumulator chamber. In some configurations, the actuator can be operatively coupled to a moment arm, a biasing structure, a diaphragm, or combination thereof to exert a force on a liquid within the accumulator chamber. For example, while the actuator is in the second state, a first end of the actuator may be arranged to deflect a first portion of the moment arm such that a second portion of the moment arm exerts a positive force against the liquid disposed within the chamber. In some such configurations, the second portion of the moment arm exerts a force on one or more intermediate components—such as an elastomeric material, hydraulic structure, diaphragm, or the like—that cooperate to transfer the force to the fluid within the chamber. In some aspects, the IMD can include an elastomer coupled to the diaphragm and movable between a compressed position and an uncompressed position, a diaphragm disposed within the chamber and movable to a pressurized state, a flexure arm having a first end disposed adjacent to the actuator and a second end adjacent to the elastomer, or combination thereof. While the actuator is in an actuated state, the second end of the flexure arm may provide a force to the elastomer, the elastomer may be in the compressed position, and the diaphragm may be in the pressurized state. In some such configurations, while the diaphragm is in the pressurized state, actuation of a valve causes a portion of the active substance within the chamber to move through the outlet into a lumen of a catheter.

In some of the present systems, the reservoir can be disposed within a cavity and the cavity can be configured to include a pressurized gas that maintains a positive pressure on a liquid within the reservoir. In some configurations, the IMD include a first valve disposed between the reservoir and an inlet of the accumulator chamber, and a second valve disposed between the outlet of the accumulator chamber and a catheter port. In some aspects, the first and second valves can be piezoelectric valves that can be controlled to deliver fluid through the IMD. A controller of the IMD can be configured to control or activate one or more components (e.g., actuator, first valve, second valve, and/or the like) to facilitate delivery of a specific dosage of pharmaceutical agent through the IMD (e.g., into a catheter for delivery to the patient). In this way and others, a specific dosage of the pharmaceutical agent can be delivered to a patient. In some aspects, the control of at least some of the components of the IMD can be performed in an open loop manner that does not rely on feedback from the fluid delivered through the IMD. For example, the controller can operate without utilizing the flowrate of the fluid exiting the IMD and, instead, utilize other parameters (e.g., pressure within chambers, time, and/or the like) to control the IMD. In this way and others, the system can include one or more processors, controllers, and sensors, all of which can be configured to communicate with one another and adjust the functioning parameters of the system via one or more signals. These control signals can be modified or associated with various applications, instructions, thresholds, and/or data sets to provide a safe and improved treatment for the patient.

The system can be configured for use with other medical accessories. For example, a syringe can be utilized to deliver an active substance to the IMD, a manifold or connectors may be utilized to ensure a secure connection between the catheter and the treatment area, and/or the like. In a non-limiting example, the system can include a multi-chamber syringe in order to facilitate the collection of cerebrospinal fluid (CSF) from the patient. The multi-chamber syringe can include one or more needles configured to be inserted into the catheter port to access the lumen, a first chamber in communication with at least one of the one or more needles, and a second chamber that is distinct from the first chamber.

In configurations in which the catheter is directly disposed within a ventricle of a patient, CSF can be more readily accessed using the present system. The needle of the multi-chamber syringe can be in fluid communication with the catheter (e.g., inserted into a catheter port), and the fluid within the lumen of the catheter can be withdrawn into the first chamber. A volume of the first chamber is greater than or equal to a measured volume of the lumen so that all of the fluid within the lumen is withdrawn. As the fluid is withdrawn into the first chamber, the lumen will be filled with CSF. When substantially all the fluid is withdrawn, the multi-chamber syringe can be used to withdraw the CSF into the second chamber. In some aspects, the multi-chamber syringe can include a lever or valve to switch fluid communication from the needle and the first and second chambers. After an adequate sample has been collected, the pharmaceutical agent can be deposited back into the lumen to continue treatment of the patient. In this way and others, the present systems enable sampling of CSF directly from the IMD, which is not available in any known systems.

In some aspects, the systems and components described herein can be utilized to perform methods or operations. Some such operations can include determining a first dosage of an active substance to be delivered to a patient. Some methods can include actuating the IMD disposed within a patient to deliver the first dosage to a ventricle of the brain via a catheter. Some of the present methods can include detection of an intracranial pressure of the ventricle of the brain via a sensor of the IMD. Some such methods can further include determining whether the intracranial pressure is greater than a pressure threshold (e.g., a hydrocephalus threshold) and, based on the intracranial pressure being greater than the pressure threshold, preventing the actuator from initiating delivery of a second dosage of the active substance. In other methods, the IMD can be configured to adjust actuation based on the intracranial pressure being within a first range or being less than another pressure threshold. In some such methods, the ranges or thresholds can be adjustable.

In other methods, some such operations can include positioning a distal end of the catheter into the central nervous system or area of resection. As a non-limiting example, the distal end of the catheter can be disposed within a portion of the body (e.g., organ or tissue) where a tumor has been removed. In some such configurations, the IMD can deliver an active substance to the portion of the body.

In some aspects, one or more methods of the present disclosure can include delivering carboplatin and/or temozolomide (TMZ) to a target area (e.g., within the brain, to the central nervous system, to an area of resection, or the like). In some such methods, an IMD can deliver carboplatin or temozolomide directly into a resected tumor site for treating cancer (e.g., glioblastoma) to increase localized drug concentration at the tumor site while decreasing systemic toxicity.

Some aspects of the present methods can include detecting a parameter with the IMD and utilizing the parameter to perform an operation. As a non-limiting example, some methods can include detecting an intracranial pressure (ICP) waveform and determining a scenario (e.g., occlusion, leak, or the like) based on the ICP waveform. Additionally, or alternatively, some methods can include determining an orientation of the IMD and determining a position of a patient (e.g., upright or prone) and utilizing the position information to determine an ICP of the patient. For example, some methods can adjust the ICP reading to account for postural changes of a patient as determined by the orientation of the IMD. Some operations include transmission of information from the IMD, such as transmitting information associated with the ICP waveform to an external device, transmitting a signal for initiating an alert at the external device (e.g., based on the orientation being outside of an inclination threshold), and/or the like. The transmission can be directly to an external device or via an intermediary device. For example, transmission of information can include transmitting information from the IMD to an intermediary via a first protocol and transmitting information from the IMD to a user device via a second protocol.

The transmission of information can be used to assist in certain procedures. To illustrate, the information associated with the ICP waveform can be utilized to determine a distal end of the catheter is positioned in a ventricle of the brain without imaging equipment. Some such determinations can be relayed to users via one or more alerts or signals from the IMD.

Some aspects of the present methods can include determining a valve actuation duration based on a pressure within a treatment area, a pressure within a reservoir, or the like. As a non-limiting example, some methods can include determining a treatment area pressure (e.g., CSF pressure), calculating an expulsion time to expel fluid within the chamber of the IMD, and setting an actuation time of a valve to be equal to the expulsion time or slightly greater than the expulsion time (e.g., 101, 105, 110, 115, 120 percent of the expulsion time or any value therebetween).

The term “coupled” is defined as connected, although not necessarily directly, and not necessarily mechanically. The terms “a” and “an” are defined as one or more unless this disclosure explicitly requires otherwise. The term “substantially” or “approximately” is defined as largely, but not necessarily wholly, what is specified (and includes what is specified; e.g., substantially 90 degrees includes 90 degrees and substantially parallel includes parallel), as understood by a person of ordinary skill in the art. In any disclosed configuration, the terms “substantially” may be substituted with “within [a percentage] of” what is specified, where the percentage includes 0.1, 1, 5, or 10 percent.

The terms “comprise” (and any form thereof such as “comprises” and “comprising”), “have” (and any form thereof such as “has” and “having”), and “include” (and any form thereof such as “includes” and “including”) are open-ended linking verbs. As a result, something that “comprises,” “has,” or “includes,” one or more elements possesses those one or more elements, but is not limited to possessing only those one or more elements. Likewise, a method that “comprises,” “has,” or “includes,” one or more steps possesses those one or more steps, but is not limited to possessing only those one or more steps.

Furthermore, all numerical ranges herein should be understood to include all integers, whole or fractions, within the range, inclusive of the ends of the ranges. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth. Additionally, the use of between when describing two endpoints of a numerical range should be understood to include those endpoints. For example, a disclosure of between 1 to 10 should be construed as supporting a range including 1 and including 10.

Herein, “or” is inclusive and not exclusive, unless expressly indicated otherwise or indicated otherwise by context, and can have the same meaning as “and/or.” To illustrate, A, B, and/or C includes: A alone, B alone, C alone, a combination of A and B, a combination of A and C, a combination of B and C, or a combination of A, B, and C. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Any configuration of any of the apparatuses, systems, and methods can consist of or consist essentially of—rather than comprise/include/have—any of the described steps, elements, or features.

The feature or features of one configuration may be applied to other configurations, even though not described, or illustrated, unless expressly prohibited by this disclosure or the nature of the configurations. Further, an apparatus or system that is configured in a certain way is configured in at least that way, but it can also be configured in other ways than those specifically described. Some details associated with the configurations described above and others are described below.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 is a schematic view of an example of the present pump system.

FIG. 2 is a schematic view of an example of a pump system of the present disclosure implanted within a patient.

FIG. 3 is a control schematic of an example of a pump system of the present disclosure.

FIG. 4 is a schematic view of an example of a pump system of the present disclosure.

FIGS. 5A and 5B are perspective views of an example of a pump system of the present disclosure, with and without the top housing, respectively.

FIG. 5C is a sectional view of the pump system taken along line A-A of FIG. 5A.

FIG. 5D is a front view, without the top and bottom housing, of the pump system of FIG. 5A.

FIGS. 6A-6C are back perspective, side, and front perspective views, respectively, of the actuator of the pump system shown in FIG. 5A.

FIGS. 6D and 6E are perspective and side views, respectively, of the actuator of FIG. 6A taken along line B-B of FIG. 6A.

FIG. 7A is a perspective view of an example of a fluid control unit of the present pump systems.

FIG. 7B is a side sectional view of the fluid control unit of FIG. 7A.

FIG. 8A is a side sectional view of an example of an actuator of the present pump systems.

FIG. 8B is an enlarged view of the accumulator shown in FIG. 8A.

FIGS. 8C-8D side sectional views of the actuator shown in FIG. 8A in first and second calibration positions, respectively.

FIG. 9A is a perspective view of another example of a pump system of the present disclosure.

FIG. 9B is a sectional view of the pump system of FIG. 9A taken along line C-C of FIG. 9A.

FIGS. 10A and 10B are top and perspective views, respectively, of an actuator of the pump system shown in FIG. 9A.

FIG. 11A is an example of external devices of the present pump systems.

FIG. 11B is a schematic diagram of an example of the present pump systems.

FIG. 12A is a perspective view of a multi-chamber syringe system which can be used with the present pump systems.

FIG. 12B shows the multi-chamber syringe system of FIG. 12A in use with an implanted pump system.

### DETAILED DESCRIPTION

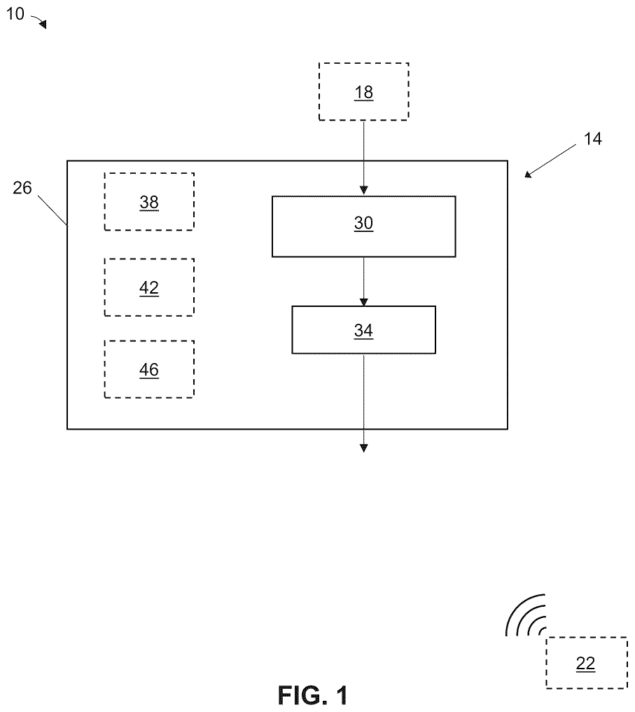

Referring now to the drawings, and more particularly to FIG. 1, shown therein and designated by the reference numeral 10 is a system for delivering fluid (e.g., therapeutics) to a subject (e.g., patient, animal, or the like), monitoring parameters of the subject, or performing treatment (e.g., surgical, non-invasive, or therapeutic) of a subject. As shown, system 10 can include an implantable medical device (IMD) 14, one or more medical accessories 18, one or more electronic accessories 22, alone or a combination thereof. The components of system 10 can be used together, or individually, to perform the operations described herein. For example, as described herein, system 10 can be configured to dispense an active substance (e.g., therapeutics or other medicine) directly to the ventricle of a subject. As should be understood, system 10 is not limited solely to delivering therapeutics to the brain of a patient and could be used in other operations (e.g., intravenous or ventricular delivery, delivery to an area of resection, or the like).

IMD 14 can be configured to deliver a fluid (e.g., pharmaceutical agent) to a target location (e.g., within a ventricle of a patient, within the central nervous system, or within an area of resection). In configurations in which fluid is delivered to the brain, IMD 14 can be configured to provide continuous or intermittent convection-enhanced delivery (CED). As shown in FIG. 1, IMD 14 can include a housing 26 that defines or otherwise forms an operating space (e.g., chambers, cavities, reservoirs, channels, lumens, or other pathways) that is configured to receive and distribute fluid though the IMD. Referring to the illustrative example of FIG. 1, IMD 14 can define a reservoir 30, an accumulator chamber 34, one or more channels in fluid communication with the reservoir or accumulator, or a combination thereof. In some configurations, reservoir 30 or chamber 34 can be in fluid communication with one or more ports (e.g., inlets or outlets). For example, reservoir 30 may be in fluid communication with an inlet (e.g., access port) and may receive fluid from an external source (e.g., medical accessory 18) that will be dispersed through IMD 14. To further illustrate, accumulator chamber 34 may be in fluid communication with an outlet (e.g., catheter port) and may transport the fluid to an external target (e.g., brain of a patient). Reservoir 30 and chamber 34 can cooperate to receive and dispense fluid from IMD 14 and can be selectively configured to transport fluid through the IMD (e.g., via one or more valves, actuators, or the like). As described herein, IMD 14 can include one or more components (e.g., cavity filled with fluid, a spring, or the like) to apply pressure to reservoir 30, chamber 34, or other passageways to move fluid through the IMD.

As depicted in FIG. 1, IMD 14 can include an actuator 38, one or more controllers 42, one or more sensors 46, or a combination thereof. Actuator 38 can be configured to cause fluid to move through (or out of) IMD 14. To illustrate, actuator 38 can be configured to cause fluid to move from chamber 34 to an outlet of IMD 14. Although not depicted in FIG. 1, actuator 38 can operate in conjunction with one or more other components (e.g., valves) to cause the transfer of fluid through or out of IMD 14. In some configurations, actuator 38 is a piezoelectric actuator that is configured to provide a mechanical force on an object in response to receiving an electrical signal. In some such configurations, actuator 38 can be a linear piezoelectric actuator that is configured to expand in a linear direction (e.g., exert a linear force on an object). Actuator 38 can be programmed or otherwise controlled to achieve the various operations described herein. For example, in at least some configurations, controller 42 can be configured to initiate movement of actuator 38 between a first state and a second state. In some such configurations, control of actuator 38 can be, but need not be, performed in an open loop manner.

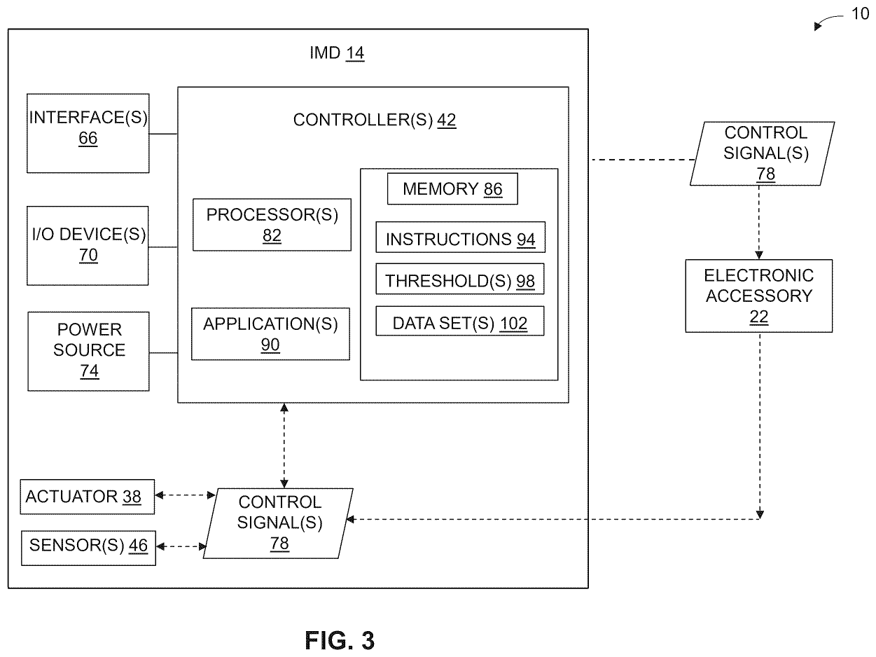

IMD 14 can include one or more electrical components (e.g., controller 42, sensors 46, or the like) that are configured to initiate various operations of system 10. As described in further detail with respect to FIG. 3 below, controller 42 and sensors 46 can interact with (e.g., communicate) each other or other components, such as electronic accessories 22, to perform programed operations. For example, controller 42 can be configured to accurately dispense a target amount of fluid to a subject such that a target dosage of the fluid can be delivered to the subject over a predetermined period of time. Sensors 46 can be configured to detect and measure various parameters associated with the operation of IMD 14. As described herein, these parameters can be used for functions independent of control of the fluid flow into the subject (e.g., target dosage). Although controller 42 is described as a single controller for brevity, system 10 can include a plurality of controllers operating together to perform the functions described herein.

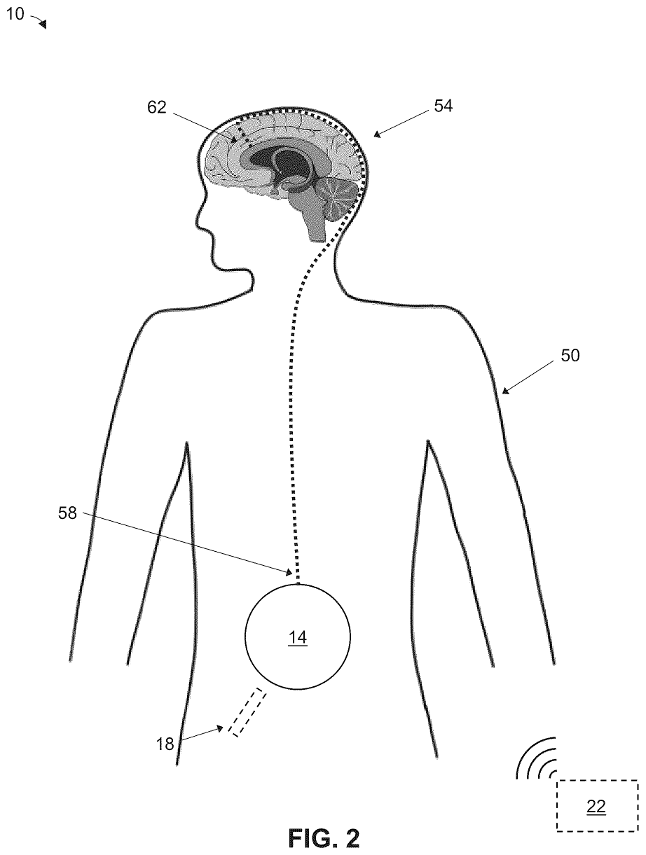

Referring now to FIG. 2, an example of system 10 is shown in use with a patient 50 to deliver fluid to a treatment area. As shown, IMD 14 can be disposed within or adjacent to the abdomen of patient 50 and configured to deliver pharmaceutical agents directly to the brain (e.g., into a ventricle or cavity thereof). The pharmaceutical agents can be delivered from IMD 14 (e.g., reservoir 30 or chamber 34) to the treatment area via a catheter 54 that defines a lumen to facilitate fluid flow. In some configurations, catheter 54 can be unitary (e.g., a single catheter extends from IMD 14 to the treatment site) or can include multiple sections that are connected together via suitable couplers or connectors. In some configurations, catheter 54 can include a proximal end 58 and a distal end 62 that may be disposed within the treatment area to deliver therapeutics to the treatment area. Proximal end 58 of catheter 54 can be coupled to IMD 14 and configured to be in fluid communication with reservoir 30, chamber 34, or both. In some configurations, such as that shown in FIG. 2, substantially all of catheter 54 may be disposed entirely within patient 50. Catheter 54 can be coupled to or utilize one or more medical accessories (e.g., 18) to assist with placing the catheter into the brain of patient 50, such as skull mounted equipment (e.g., tubes, manifolds, needles, lumens, drills, or the like).

System 10 enables continuous delivery of therapeutics directly to a ventricle of the brain of patient 50 that is not available in conventional operations, in which therapeutics are intermittently delivered into an Omaya reservoir via a syringe. Further, system 10 allows for accurate delivery of therapeutics to the brain that cannot be achieved when delivering therapeutics indirectly to the brain via CSF. Thus, system 10 is designed to achieve certain functionality not available for indirect delivery systems. For example, in the configuration shown in FIG. 2, a pressure sensor (e.g., 46) can be disposed within IMD 14 and in fluid communication with catheter 54 to enable detection of an intracranial pressure (ICP) waveform when distal end 62 of the catheter is disposed directly within a ventricle of the brain. In some configurations, IMD 14 is configured to detect a gauge pressure of the treatment area, which is not available with conventional implantable pumps. As described herein, system 10 and IMD 14 provide a manner in which ICP can be monitored in real time outside of an operating room, a procedure that can typically prove both invasive, costly, and constraining to a patient. In some configurations, IMD 14 can include other sensors, such as an accelerometer (e.g., 46) disposed within IMD 14 to determine an orientation of patient 50, a temperature sensor, or the like. Additionally, or alternatively, an electrode may be disposed at distal end 62 of catheter 54 and IMD 14 can include an electrical sensor to monitor parameters at the electrode (e.g., current).

Information associated with the ICP waveform can be recorded or transferred to an external device, such as an electronic accessory 22. In at least this way, ICP measurement, ICP waveforms, and other vital parameters associated with ICP can measured (e.g., via sensor 46) and then monitored, modified, or transmitted (e.g., via controller 42) to assist with treatment of a patient 50. Further, as IMD 14 can remain in a patient 50 for a prolonged period, system 10 provides a mechanism to monitor ICP outside of a hospital (e.g., at home), which is not available in conventional ICP monitoring techniques. Accordingly, system 10 provides patient 50 with a much greater degree of freedom than currently exists. As described in further detail below, system 10 can utilize this information in various ways (e.g., to adjust a dosage, to skip a dosage, to send alerts to patient 50, to send alerts to medical professionals, to record patient data, or the like).

Although FIG. 2 depicts the treatment area as being within the ventricle of the brain, it should be understood that system 10 or IMD 14 may be used for other treatment applications. For example, in some configurations a distal end 62 of catheter may be positioned at or adjacent to an area of resection. In some such configurations, the area of resection can be a portion of an organ or tissue where a tumor has been removed. In other configurations, the treatment area can include other portions of the central nervous system, blood stream, nerves, tissues or the like.

Referring now to FIG. 3, shown is a schematic diagram of system 10. As depicted, IMD 14 can include controller 42 configured to control one or more operations of system 10, such as, but not limited to, operation of the flow of fluid through the IMD, operation of valves, operation of ports, actuation of elements, monitoring of flow parameters, therapeutic concentrations, pressures, temperatures, electric currents, or the like. Controller 42 can include a single controller or multiple controllers cooperating together to perform the functions described herein. Controller 42 can be in wired or wireless communication with various components of system 10. In the depicted configuration, IMD 14 may comprise one or more interface(s) 66, one or more I/O device(s) 70, and a power source 74 coupled to controller 42. System 10 can include one or more sensor(s) 46 configured to detect one or more parameters and to provide data to controller 42 (e.g., via control signals 78). Actuator 38 can be configured to be actuated by controller 42 (e.g., via control signals 78). In some configurations, circuitry (e.g., a PCB, wires, etc.) may connect components of IMD 14 with one or more other components of system 10.

Controller 42 may include a processor 82 coupled to a memory 86 (e.g., a computer-readable storage device). In some configurations, controller 42 may include one or more application(s) 90 that access processor 82 and/or memory 86 to operate IMD 14 and/or other components of system 10. Processor 82 may include or correspond to a microcontroller/microprocessor, a central processing unit (CPU), a field-programmable gate array (FPGA) device, an application-specific integrated circuits (ASIC), a system-on-chip (SOC), a single-board computer system (SBC) (such as, for example, a computer-on-module (COM) or system-on-module (SOM)), another hardware device, a firmware device, or any combination thereof. Memory 86, such as a non-transitory computer-readable storage medium, may include volatile memory devices (e.g., random access memory (RAM) devices), nonvolatile memory devices (e.g., read only memory (ROM) devices, programmable read-only memory, and flash memory), or other types of integrated circuits (ICs) (e.g., field-programmable gate arrays (FPGAs) or application-specific ICs (ASICs)), or any other suitable computer-readable non-transitory storage media. Memory 86 may be configured to store instructions 94, one or more threshold(s) 98, one or more data set(s) 102, or a combination thereof.

In some configurations, instructions 94 (e.g., control logic) may be configured to, when executed by the one or more processor(s) 82, cause the processor(s) to perform one or more operations (e.g., actuate valves, actuate actuator 38, send detected parameters, send alerts, compare values, or the like). The one or more thresholds 98 and one or more data sets 102 may be configured to cause the processor(s) 82 to generate control signals (e.g., 78). For example, the processor(s) 82 may initiate and/or perform operations as described with reference to FIGS. 1-11B. As a specific example, thresholds 98 can include a maximum or minimum patient parameter (e.g., pressure within catheter, pressure at target area, electrical current, heartrate, or the like), a maximum or minimum dosage or other fluid parameter (e.g., flowrate), or threshold associate with any other parameter monitored or utilized by system 10 (e.g., parameters within IMD). Thresholds 98 can also include timing thresholds such as a duration a valve should be opened, a time between actuation of a component (e.g., valves, actuators, or the like), or the like. Data set(s) 102 can include data associated with thresholds 98 or other parameters of system 10, such as, data (e.g., chemical concentrations, pressures, temperatures, flow rates, or the like) detected by sensors 46. For another example, processor 82 can compare an ICP of the patient to a pressure threshold 98 and prevent actuator 38 from initiating delivery of a fluid (e.g., pharmaceutical agent) should the ICP be higher than the threshold. As an illustrative, non-limiting example, the thresholds can correspond to a state of hydrocephalus. In some of the configurations described herein, pressure thresholds can utilize gauge pressures, rather than absolute pressures.

Application(s) 90 may communicate (e.g., send and/or receive) with processor 82 and memory 86. For example, application(s) 90 may receive data from sensor(s) 46 or memory 86 (e.g., data sets 102), manipulate the data, and send a signal to processor 82 to cause the processor to transmit the manipulated data (e.g., via interface(s) 66) or store the manipulated data (e.g., via memory 86). In some configurations, controller 42 is configured to generate and send control signals 78 to an external device (e.g., electronic accessory 22). For example, controller 42 may generate and/or send control signals 78 responsive to receiving a signal and/or one or more user inputs via the one or more interface(s) 66 and/or the one or more I/O device(s) 70.

Interface(s) 66 may include a network interface and/or a device interface configured to be communicatively coupled to one or more other devices. For example, interface(s) 66 may include a transmitter, a receiver, or a combination thereof (e.g., a transceiver), and may enable wired communication, wireless communication, or a combination thereof, such as with I/O device(s) 70. In some configurations, interface(s) 66 can include a long range (LoRa) interface, a Wi-Fi interface (e.g., an Institute of Electrical and Electronics Engineers (IEEE) 802.11 interface), a cellular interface (e.g., a fourth generation (4G) or long term evolution (LTE) interface, a fifth generation (5G) new radio (NR) interface, or the like), a Bluetooth interface, a Bluetooth low energy (BLE) interface, a Zigbee interface, a MICS, a personal area network (PAN), a local area network (LAN), a wide area network (WAN), a metropolitan area network (MAN), or one or more portions of the Internet, another type of network interface, or the like. I/O device(s) 70 can include one or more switches (e.g., depressible buttons, triggers, or the like), one or more speakers, one or more light sources, vibration devices, or other types of devices that enable a user to receive information from or provide information to controller 42. In some configurations, interfaces(s) 66 and/or I/O device(s) 70 may enable a wired connection to controller 42 via a port or other suitable configuration. Power source 74 may be coupled to controller 42, interface(s) 66, I/O device(s) 70, sensor(s) 46, actuator 38, or a combination thereof. In some configurations, power source 74 may be coupled to components of IMD 14 via circuitry. In some configurations, power source 74 may include a battery, capacitors, a charge storage device, or the like.

Sensor(s) e.g., 46 may be coupled to one or more components of system 10 (e.g., catheter 54 or IMD 14). In some configurations, sensor(s) 46 are configured to determine parameters associated with fluid flow within IMD 14. For example, sensor(s) 46 can be configured to measure physical properties of the fluid (e.g., density, viscosity, temperature, pressure, specific weight, specific volume, specific gravity, or the like), surface tension, pulsatility, fluid pressure, fluid flow rate, fluid velocity, or the like. In some aspects, sensor(s) 46 may comprise one or more of a flow meter, a MEMS sensor, an in-line fluidic sensor, a pressure sensor, a temperature sensor, mass flow sensors, ultrasonic sensors, a level sensor, an infrared sensor, an accelerometer, a current sensor, electrodes (e.g., for an electroencephalogram, electromyography or the like), humidity sensor, timers, chemical sensors to determine therapeutic concentration in a treatment area (e.g., CSF), a gyroscope, a level sensor, a capacity sensor, a photoelectric sensor, or other suitable sensors, or combination thereof.

In some configurations, instructions 94 (e.g., control logic) may be configured to, when executed by the one or more processors 82, cause the processor(s) to perform one or more operations. For example, the one or more operations may include receiving a message (e.g., control signal 78, a command, or an instruction) to perform an operation. In some configurations, the one or more operations may include transmitting a message (e.g., control signal 78, a command, or an instruction). To illustrate, the operation may include measuring (e.g., via sensor 46) an ICP of a patient (e.g., 50). To further illustrate, the operation may include initiating actuation of, or adjusting a rate of actuation of, actuator 38. In some such configurations, the operation may include initiating actuation of one or more valves (e.g., piezoelectric valves) in conjunction with actuation of actuator 38 to cause IMD 14 to pump a fluid at a specific flowrate, or within a specified range of flowrates, or a specific volume of fluid. Valves may be controlled based on other measured parameters, thresholds, data, or the like. For example, valves can be controlled to be actuated for minimal time durations, which can help prolong the useful life of the valves and minimize power use. In some such configurations, the duration a valve is actuated can be controlled or adjusted based on one or more pressure readings, as described herein.

In some configurations, the one or more operations may include initiating movement of actuator 38 between the first state and the second state. In some such configurations, initiating movement of actuator 38 can be performed in an open loop manner. In some such operations, the flow rate (or other fluid parameters) of fluid downstream of IMD 14 is not utilized as feedback to control or adjust actuator 38. In some configurations, actuator 38 can be controlled or adjusted based on an ambient pressure, pressure within reservoir 30 or other passages or containers, the volume of chamber 34, the displacement distance of accumulator 106, or a combination thereof.

The one or more operations may also include transmitting signals (e.g., information, alerts, or the like) to an external source (e.g., electronic accessory 22). For example, controller 42 may be configured to initiate transmission of signals or information between different components of system 10. To illustrate, IMD 14 can transmit information measured by the sensors 46 (e.g., ICP), information derived from measurements, alerts indicative of a target scenario (e.g., confirmation of placement of distal tip within target area of the brain, ICP below minimum threshold, or the like), or other relevant signals. In some configurations, the one or more operations may include detecting a position of patient 50 via a sensor 46 (e.g., an accelerometer) within IMD 14 and performing one or more actions based on the detected position corresponded to a target position, such as when the patient is laying down or in a prone state. For example, accurate measurement of a pressure of the target area can be improved by having a patient (e.g., 50) lie down (such that the target area (e.g., ventricle) is at substantially the same height as IMD 14) to equalize the pressure throughout the system 10. The patient (e.g., 50) can be instructed to lie down via an alert sent to an external source (e.g., displayed at electronic accessory 22). The measurement while the patient is in a pre-determined orientation can be stored in memory and configured to establish a baseline. In some configurations, various other measurements can be recorded at other orientations to determine a relationship between the measurement (e.g., pressure) and patient orientation. Additionally, or alternatively, controller 42 can be configured to calculate a pressure estimate at various orientations to determine a relationship between the pressure and patient orientation and, in some configurations, can utilize variable input by the user such as a density of the fluid within the reservoir, a distance between IMD 14 and the treatment area, or the like.

In some such operations, IMD 14 can be configured to alter pressure readings based on patient orientation, such as by altering one or more thresholds. For example, a first pressure threshold may be adjusted higher while the patient is in a vertical orientation, adjusted to be lower while the patient is in a horizontal orientation, or both. In this way and others, IMD 14 is configured to adjust the ICP reading to account for postural changes of a patient as determined by the orientation of the IMD. In some configurations, system 10 may issue alerts based on patient orientation. To illustrate, the operations may include determining an orientation of IMD 14 and based on the orientation being outside of an inclination threshold, initiating an alert to be sent to an external device (e.g., 22). Additionally, or alternatively, based on the orientation being within the inclination threshold, IMD 14 can determine the intracranial pressure.

The one or more operations may also include determining distal end 62 of catheter 54 is positioned in a treatment area (e.g., ventricle) of the brain based on information associated with the ICP waveform. In some such configurations, IMD 14 can initiate an alert to be sent to an external device (e.g., 22) based on the determination that distal end 62 of catheter 54 is positioned in the treatment area. In this way, and others, IMD 14 can provide an indication that the catheter is correctly placed without other equipment, such as imaging devices (e.g., x-rays and MRIs).

In some configurations, the operations can include detecting a leak or occlusion within catheter 54. Some such operations can include detecting a signature associated with the leak or occlusion via the pressure sensor. In some configurations, the signature can correlate to a dampened ICP waveform or a change in the shape of the ICP waveform, or be based on one or more thresholds, or a combination thereof. Additionally, or alternatively, some operations can include detecting an electrical current at the target area (e.g., via an electrode at a distal end of catheter). The current can be compared to a baseline threshold to determine if an abnormality is present, such as a higher electrical current that is associated with a seizure. In the present operations, IMD 14 can be configured to transmit a message (e.g., alert) to an electronic accessory (e.g., 22) when one or more of the observed properties exceed a threshold, match a signature, or the like.

In some configurations, the operations described herein can be performed in an automated manner. For example, controller 42 can be configured to receive signals and initiate transmission of signals to control IMD 14 to function as described herein. In this way and others, actuators, valves, and other components of system 10 or IMD 14 can be actuated or otherwise manipulated to operate in the variety of manners described herein.

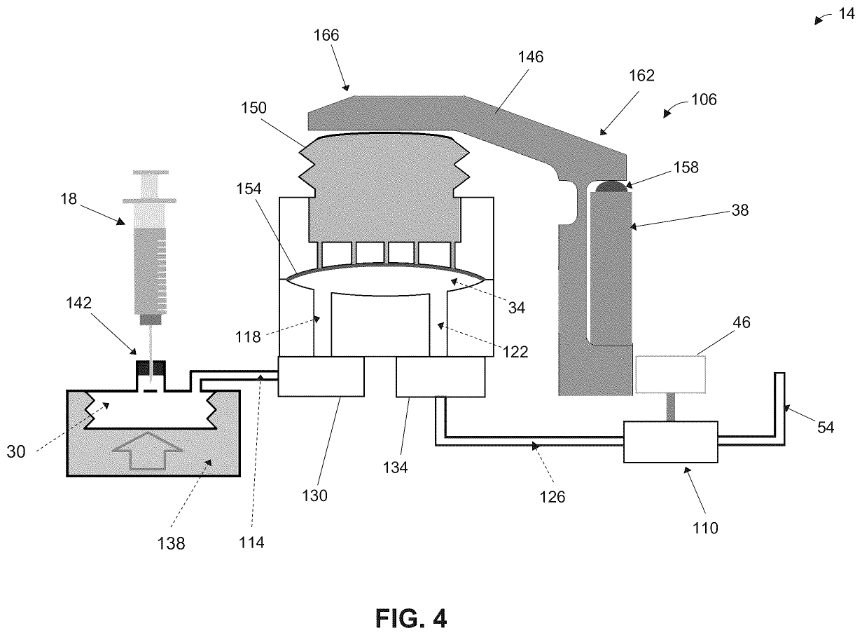

Referring now to FIG. 4, another schematic illustration of system 10 is shown. As illustrated, system 10 includes IMD 14, which can receive fluid from a medical accessory 18 (e.g., a syringe). IMD 14 can include an accumulator 106 and define multiple avenues or flow paths (e.g., chambers, reservoirs, or channels and associated inlets and outlets) for the fluid to flow through the IMD, and these avenues can be defined by the various structures of the IMD (e.g., components of housing 26, accumulator 106, bodies, covers, manifolds, or the like). For clarity, the below description is focused on the flow paths, but it should be understood that IMD 14 or housing 26 includes the structures defining these flow paths (e.g., pipes, hoses, conduits, tubing, vessels, containers, receptacles, tanks, repositories, base, bodies, or the like).

As shown in FIG. 4, IMD 14 defines reservoir 30 that is in fluid communication with chamber 34 that is defined by accumulator 106 and can be in fluid communication with a catheter port 110 via a plurality of fluid channels (e.g., 114, 118, 122, 126). The flow of fluid through reservoir 30, chamber 34, and catheter port 110 can be controlled by various components of IMD 14, as described herein. In some configurations, IMD 14 includes a first valve 130 disposed between reservoir 30 and chamber 34 and a second valve 134 disposed between chamber 34 and an outlet, for example, catheter port 110. Each valve can be actuated between an open state, in which fluid flow is permitted through the valve, and a closed state, in which fluid flow through the valve is blocked. In the illustrative example shown in FIG. 4, IMD 14 defines a flow path from reservoir 30, through a first channel 114, through first valve 130, through a second channel 118, through chamber 34, through a third channel 122, through second valve 134, through a fourth channel 126, and to catheter port 110. In some configurations, catheter 54 is connected to catheter port 110 to facilitate delivery of the fluid (e.g., into the ventricle of the brain of patient 50, intermittent CED, continuous CED, or the like).

In some configurations, reservoir 30 can be contained within a pressurized cavity 138 that is defined by IMD 14 (e.g., by housing 26). Cavity 138 can be filled with a fluid (e.g., gas) such that the fluid provides a positive pressure on reservoir 30 to pressurize the fluid within the reservoir. In an illustrative, non-limiting example, cavity 138 may be filled with Chlorofluorocarbons (CFCs) (e.g., Freon), Hydrochlorofluorocarbons (HCFCs), or other a heat sensitive saturated gases (e.g., gases including hydrocarbons, HFOs (hydrofluoroolefins), HFCs (hydrofluorocarbons), CO2 (carbon dioxide), or ammonia). Additionally, or alternatively, a spring (e.g., wave spring, belleville spring, disc spring or the like) can be disposed within cavity 138 cand configured to engage reservoir to exert a pressure on the fluid with the reservoir. The spring (not shown) may be sufficiently small to be positioned between the housing and a bottom wall of the reservoir.

Reservoir 30 can additionally define an access port 142 to allow for the delivery of a fluid (e.g., pharmaceutical agent) into the reservoir. Access port 142 can include a penetrable membrane to selectively enable delivery of fluid into reservoir 30. To illustrate, a needle of a syringe can penetrate the membrane to deliver/withdraw fluid while mitigating fluid leakage from the membrane after the needle has been removed. In at least this way, a medical accessory 18 (e.g., a syringe) can penetrate access port 142 to facilitate the delivery of a fluid into reservoir 30.

First valve 130 can be connected to chamber 34 (e.g., by second channel 118) to either allow or restrict fluid communication from reservoir 30 to the chamber. First valve 130 may be, for example, a piezoelectric valve that can be selectively opened to allow a desired amount of fluid into chamber 34 and retroactively closed to restrict fluid flow into the chamber after a desired dosage of the fluid flows through the valve. In some configurations, first valve 130 can be opened until chamber 34 is filled with a target volume of fluid (e.g., 100 percent of the volume of chamber 34). In some such configurations, chamber 34 is completely filled with fluid. First valve 130 may be controlled via controller 42 and can be controlled based on a timer or other parameters (e.g., pressures). For example, a duration of actuation of first valve 130 may be adjusted based on a pressure acting on reservoir 30 so as to open the first valve for a minimum amount of time (e.g., valve is controlled to close once chamber 34 is completely filled with fluid). In some configurations, controller 42 can adjust the duration of actuation of first valve 130 if the pressure acting on reservoir 30 changes (which changes the time it takes chamber 34 to be filled with fluid).

Accumulator 106 can be configured to displace a fixed volume of fluid per cycle and the timing of the cycle can be set (or altered) to correspond to a target dosage. As described herein, this cycle timing can be adjusted according to one or more operations that may be pre-programmed (e.g., via instructions in IMD 14), performed by a user (e.g., via signals from an electronic accessory 22), or the like. As shown, first valve 130 can be used to restrict the flow of a fluid from reservoir 30 and first channel 114 to second channel 118 and chamber 34. As previously discussed, cavity 138 can be pressurized such that a positive pressure is exerted on fluid within reservoir 30 and first channel 114. Therefore, actuation of first valve 130 can allow the pressurized fluid to flow into second channel 118 and chamber 34 whenever selectively opened. In such configurations, the pressure within chamber 34 and second channel 118 can be maintained below the pressure of fluid within reservoir 30 or first channel 114, as this pressure differential can facilitate the delivery of a fluid from the higher-pressure reservoir to the lower-pressure chamber.

Second valve 134 can connect chamber 34 with an outlet (e.g., catheter port 110), selectively opening and closing as desired to facilitate the delivery of a fluid outside of IMD 14 (e.g., to patient 50). In some configurations, second valve 134 can be controlled in the same manner as discussed with respect to first valve 130, above. Accumulator 106, described in further detail below, can be selectively operated to increase the pressure within chamber 34. Thus, chamber 34 can be used in a similar manner to reservoir 30, in which a positive pressure can be utilized to facilitate the delivery of a fluid through IMD 14. To illustrate, first valve 130 can be opened until a target volume (e.g., desired dosage) of fluid enters into chamber 34 (through the method previously described). First valve 130 can then be closed, sealing the target volume of fluid within chamber 34 while second valve 134 is in a closed state. Second valve 134 can be opened once the first valve 130 in is the closed state to enable fluid flow to catheter port 110. Accumulator 106 can be used to increase the pressure in chamber 34 to encourage fluid flow from the chamber through third channel 122, second valve 134, fourth channel 126, and to catheter port 110. Once the fluid reaches catheter port 110 it can be further delivered to the target area of patient 50 by means of catheter 54. This process can be cycled several times per second, allowing controlled delivery of the fluid to the treatment area.

In some configurations, second valve 134 can be controlled based on a downstream pressure (e.g., pressure within target area or catheter). To illustrate, an amount of time it takes for fluid to be completely dispersed from chamber 34 (e.g., via a pressure from accumulator 106) can increase if a pressure within the treatment area increases. Thus, the time it takes to expel a fluid from chamber 34 can vary depending on the pressure within the treatment area. In some configurations, controller 42 can determine a pressure within the treatment area, as described herein, and determine a delivery time duration corresponding to the fluid being expelled from chamber 34. In some such configurations, controller 42 can actuate second valve 134 for a duration that is substantially equal to (e.g., 100, 105, 110, 115, or 120 percent of) the delivery time duration. In this way or others, the second valve 134 can close shortly after all fluid is expelled from chamber 34. In this way and others, the valves can be controlled so as to be actuated for a minimal amount of time to reduce power consumption and improve battery life of IMD 14.

In some configurations, the valves (e.g., 130, 134) are configured to be actuatable between an open state in which fluid can flow through the valve and a closed state in which fluid is restricted from flowing through the valve. The valves (e.g., second valve 134) can be controlled to deliver an active substance by switching the valve from the closed state to the open state and back to the closed state, where fluid is delivered during the time period when the valve is in the open state. The time period the valve is in the open state (e.g., delivery time) can be calculated or altered based on the pressure within the catheter or the pressure at the target area. For example, the delivery time can be increased when a pressure exceeds one or more pressure threshold or decreased when the pressure is below one or more pressure thresholds. In some configurations, a power source (e.g., 74) is configured to deliver an electrical current to the valves (e.g., 130, 134) to actuate the valves to the open state. In some such configurations, the power source can be controlled (e.g., via controller) to deliver power for a time period that is substantially equal to the delivery time.

To facilitate the pressure change of chamber 34, accumulator 106 can include actuator 38, flexure arm 146, elastomer 150, diaphragm (or membrane) 154, or combination thereof. At least some of the components of accumulator 106 are movable between different positions or states to cause fluid flow through IMD 14. For example, actuator 38 can be utilized to move flexure arm 146 such that an end of the flexure arm exerts a force within chamber 34. As depicted, flexure arm 146 can indirectly pressurize chamber 34 by exerting a force to elastomer 150 that is connected to diaphragm 154 that is coupled to chamber 34. In such configurations, the pressure applied to elastomer 150 can expand diaphragm 154 (e.g., to a pressurized state), constricting chamber 34 and increasing the pressure therein. However, it should be understood that in other configurations, actuator 38 could increase pressure in chamber 34 in a more direct manner. Thus, actuation of actuator 38 can be used to facilitate the expansion and contraction of diaphragm 154, thereby increasing or decreasing the pressure of chamber 34.

Actuator 38, (e.g., a linear actuator) can operate by means of linear piezoelectric element(s) 158, selectively movable between an actuated state and an unactuated state along a linear path. For example, an electrical current can be applied to piezoelectric element(s) 158 to either expand or contract the material, the movement of which can be used to actuate flexure arm 146. In this way, actuator 38 can be moved (e.g., via control signal(s) 78 from a controller 42) between an actuated and an unactuated state. As described herein, actuator 38 and accumulator 106 do not rely on pressurized gas to deliver fluid from chamber 34 like delivery of fluid from reservoir 30. While actuator 38 is in the actuated state, the actuator may exert a force on a first portion 162 of flexure arm 146 disposed adjacent to the actuator such that a second portion 166 of the flexure arm is moved to cause an increase in pressure within chamber 34. For example, while actuator 38 is in the actuated state, the second portion 166 of flexure arm 146 can exert a positive force on elastomer 150, thereby displacing the elastomer and causing diaphragm 154 to expand. Elastomer 150 can be a substantially non-compressible material that will be displaced when a force is exerted by flexure arm 146. Diaphragm 154 can then be displaced by elastomer 150 to force the fluid out of accumulator chamber 34. If fluid was in the sealed chamber, it would be dispensed (e.g., through second valve 134) to catheter port 110 of IMD 14. Although not shown, IMD 14 can include one or more filters (e.g., bacterial mesh filters) withing the flow path. As a non-limiting example, filters can include a first filter disposed between reservoir 30 and first valve 130 and a second filter disposed between second valve 134 and catheter port 110 (e.g., at the catheter port).

Processor (e.g., 82) can be configured to actuate actuator 38, first valve 130, and second valve 134 to deliver a dose of the fluid (e.g., pharmaceutical agent) to a treatment area in a metronomic manner. Catheter port 110 can additionally comprise sensors 46 (e.g., a pressure sensor) to monitor the intracranial pressure of the patient (e.g., 50) and send information to any connected devices (e.g., I/O devices 70, processor 82, and/or memory 86). In some configurations, a pressure sensor disposed at catheter port 110 can be isolated from other components of IMD 14 (e.g., reservoir 30, accumulator 106, or the like) such that the pressure sensor includes a direct line to a distal end of catheter 54 for undisturbed measurements. In at least some configurations, actuator 38 can be actuated (e.g., via control signal(s) 78 from a controller 42) between an actuated and an unactuated state in an open-loop manner. In such configurations, actuator 38 may be controlled independent from a flow rate of fluid within catheter 54 or fourth channel 126. That is the actuation (e.g., timing) of actuator 38 along with first and second valves 130, 134 can be performed without using the downstream flow rate of the fluid. In some configurations, actuator 38, first valve, 130, or second valve 134 can be controlled based on a predetermined time interval that corresponds with a target dosage. To illustrate, a dosage cycle (e.g., opening and closing of first valve 130, engaging and disengaging actuator 38, opening and closing second valve 134) can be set to deliver a fixed volume of fluid and processor 82 can be configured to adjust how many cycles are performed over a time period to adjust the target dosage.

In some configurations, IMD 14 can be configured to deliver therapeutics based on one or more physiological parameters. For example, IMD 14 can be configured to time or dispense therapeutics based on a specific pressure at the treatment area or distal end 62 of catheter 54. In some such configurations, detected pressure may fluctuate and IMD 14 can be configured to dispense therapeutics (e.g., open second valve 134 and engage actuator 38) at a time when the pressure is below a fluctuation threshold. In an illustrative non-limiting example, detected pressure at the treatment area may fluctuate cyclically based on a heartbeat of the user and IMD 14 can be configured to measure this pressure fluctuation and adjust the timing of actuation of components (e.g., valves and actuator) to dispense therapeutics when the pressure at the treatment site is at or near its lowest pressure (e.g., diastolic). For example, IMD 14 can detect (e.g., via pressure sensor 46) reoccurring periods of high pressure and low pressure and dispense therapeutics during the detected periods of low pressure. Additionally, or alternatively, pressure can be increased based on other non-cyclical events such as a patient sneezing, coughing, or the like. IMD 14 can be configured to adjust delivery timing based on these high pressure events to avoid dispensing therapeutics at a time of high pressure. Timing of the therapeutics delivery based on pressure fluctuation can improve accuracy of dosing to the patient that is not available in current medical pumps or medical devices that provide pressure readings based on average detected pressure. IMD 14 can be configured to adjust other operations described herein based on the detected pressure changes or associated thresholds, such as by not performing certain operations during a first period or altering the operations to occur during a second period. For example, pressure readings of the treatment area (e.g., ICP) can be measured during a specific period (e.g., period of low pressure). In some configurations, only readings associated with a specific period are sent to the user or utilized for other operations or calculations described herein.

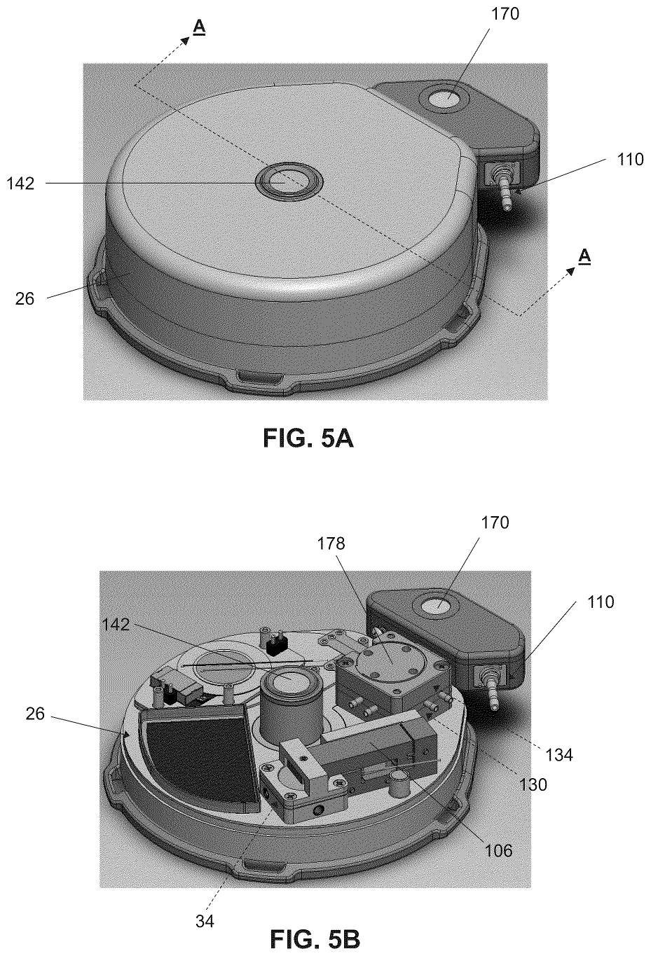

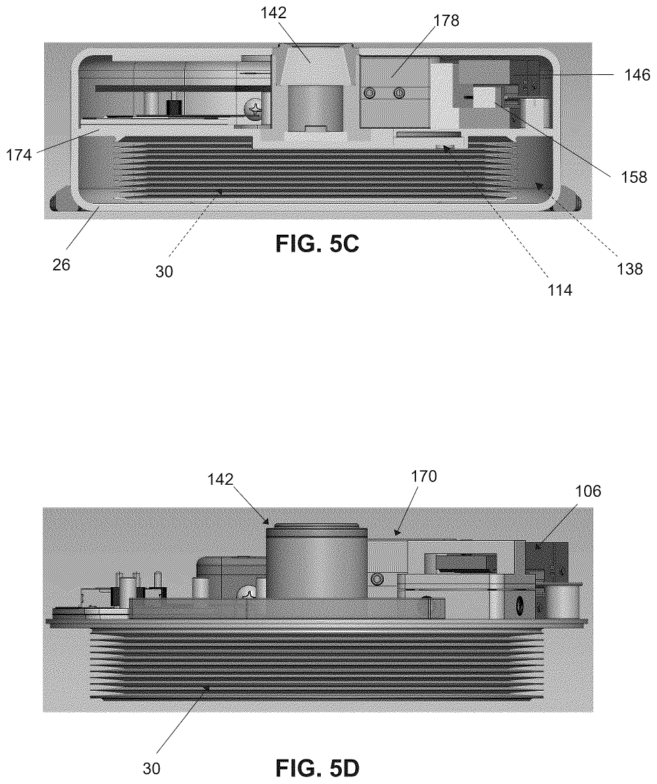

Referring now to FIGS. 5A-5D, an example of IMD 14 is shown. FIGS. 5A and 5C depict IMD 14 with housing 26 fully installed on the device and FIGS. 5B and 5D depict the IMD 14 with the top portion of housing 26 removed, illustrating various internal components. The example of IMD 14 illustrated in FIGS. 5A-5D can operate similarly to the schematic example shown in FIG. 4. As described above, access port 142 can be penetrated by a medical accessory 18 (e.g., a syringe) to fill reservoir 30 with fluid (e.g., pharmaceutical agent). As shown in FIG. 5A, access port 142 can be accessible through housing 26 (e.g., via an opening defined in a top portion of the housing). This can allow a physician to interact with access port 142 while IMD 14 is disposed within patient 50, such as to inject a fluid into reservoir 30. In some configurations, IMD 14 can include a second access port 170 in fluid communication with catheter port 110. Similar to access port 142, second access port 170 can facilitate access to any fluid contained within the lumen of catheter 54, such as via a medical accessory (e.g., 18).

As best shown in FIG. 5C, access port 142 can extend through a separation plate 174 that divides two portions (e.g., upper and lower) of housing 26. As illustrated, reservoir 30 is disposed within cavity 138 that can be defined by a bottom portion of housing 26 and separation plate 174. In some configurations, reservoir 30 is an expandable reservoir with a volume that can vary depending on the interior and exterior pressure. For example, reservoir 30 can be defined by sidewalls that can vary in length, such as the accordion-type walls shown in FIGS. 5C and 5D. In some such configurations, the fluid properties of a liquid within the expandable reservoir 30, in combination with the fluid properties of a gas held within cavity 138, can facilitate a positive pressure on the liquid contained within the reservoir. Thus, as fluid is delivered from reservoir 30 to downstream components of IMD 14 (e.g., the reservoir is partially filled), a positive pressure can be maintained on the reservoir. Although not shown, some configurations may include a spring disposed within cavity 138. For example, the spring may include a wave spring or a belleville spring coupled to a wall of the reservoir 30 (e.g., a spring positioned between a bottom portion of housing 26 and the reservoir.

As shown in FIGS. 5B-5D, IMD 14 can include accumulator 106 and a flow control unit (FCU) 178 that can operate to transport a fluid through the IMD 14. In some configurations, accumulator 106 and FCU 178 can be disposed on or coupled to separation plate 174. First and second channels 114, 118 can be in fluid communication with accumulator 106 and FCU 178 to facilitate the flow of fluid from reservoir 30 to chamber 34. For example, FCU 178 may include first valve 130 that is operably coupled to first and second channels 114, 118. First valve 130 can be configured to operate between an open and closed state, such that, when in the open state, fluid communication is permitted between first channel 114 and second channel 118 and, when in the closed state, fluid communication is restricted between the first and second channels. In some configurations, like that shown in FIG. 5C, first channel 114 can be at least partially defined by separation plate 174 or, alternatively, connected to reservoir 30 via an opening in the separation plate.

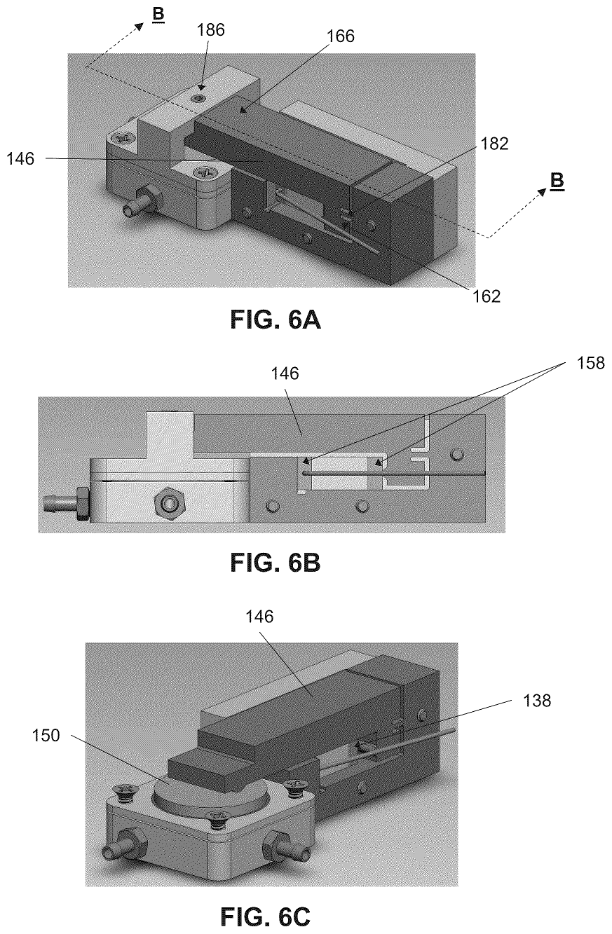

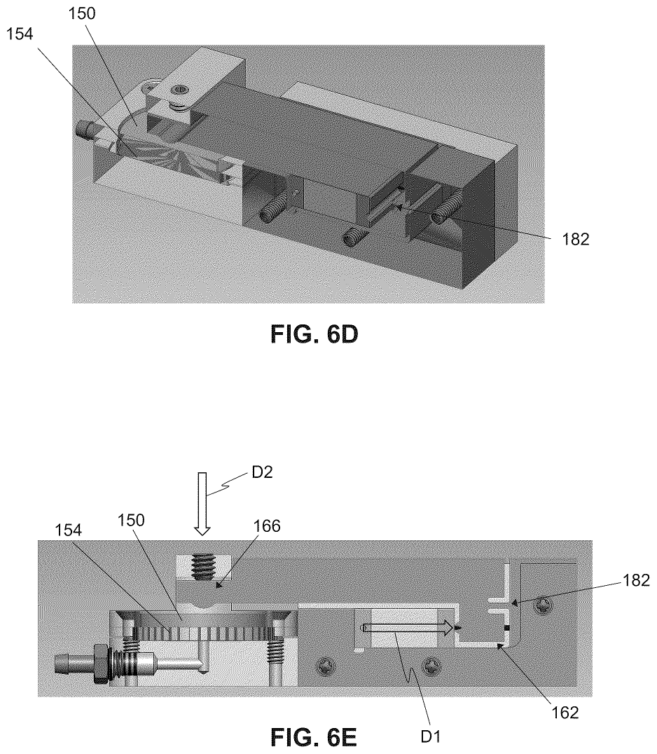

Referring to FIGS. 6A-6E, various view of accumulator 106 are shown. Accumulator 106 can define chamber 34 where fluid can be temporarily stored, such as after flowing through first channel 114, first valve 130, and second channel 118. Chamber 34 can additionally be in fluid communication with third channel 122, thereon connected to second valve 134 and fourth channel 126. As described previously, accumulator 106 can act to increase the pressure on a fluid contained within chamber 34. For example, actuator 38 can be controlled to exert a force on flexure arm 146 to cause the flexure arm 146 to exert a force on the fluid within chamber 34. For example, second portion 166 of flexure arm 146 can exert a force on elastomer 150, where the pressure on the elastomer can cause an increase in pressure in chamber 34. Second valve 134 can be actuated to an open state (in which fluid communication is permitted between third channel 122 and fourth channel 126) during this operation such that fluid can flow from chamber 34 to catheter port 110. Second valve 134 can act in a similar manner to first valve 130, in which it can operate between an open and closed state where the first state can permit fluid communication between third channel 122 and fourth channel 126 while the closed state can restrict fluid communication between the third channel and fourth channel.

In some configurations, actuator 38 can include one or more piezoelectric element(s) 158, each of which can be selectively movable between an actuated state and an unactuated state. For example, an electric current can be supplied to expand the piezoelectric element(s) 158 in a first direction D1 to exert a force on first portion 162 of flexure arm 146. The force on first portion 162 of flexure arm 146 can cause a rotation of the flexure arm about pivot point 182 such that a second portion 166 of the flexure arm exerts a force on elastomer 150 in a second direction D2. In some configurations, the first and second directions (e.g., D1, D2) are orthogonal to one another. The force on the elastomer 150 can act to increase the pressure inside of chamber 34. For example, the force can act on elastomer 150, thereby expanding diaphragm 154 to constrict chamber 34 and increase the pressure therein. In some configurations, diaphragm 154 can limit a movement of a chamber wall or elastomer in a certain direction (e.g., toward or away from second direction D2) .

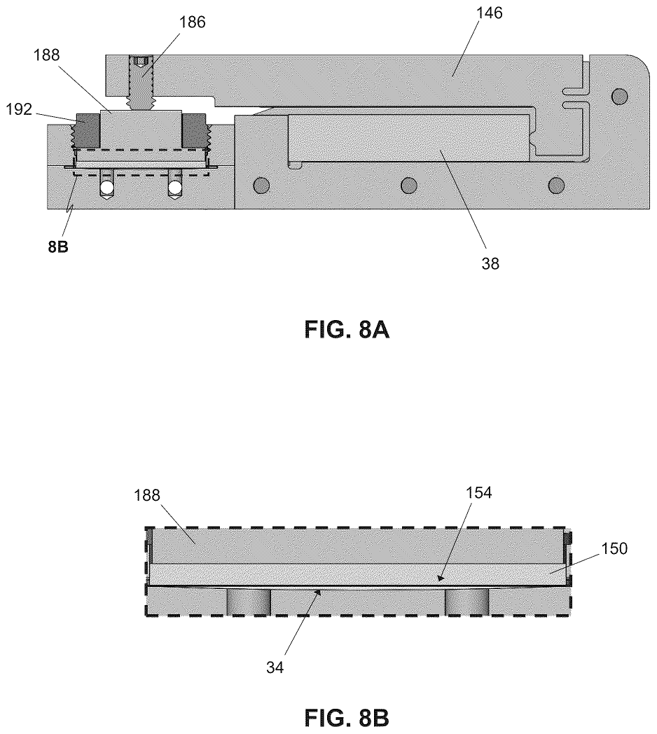

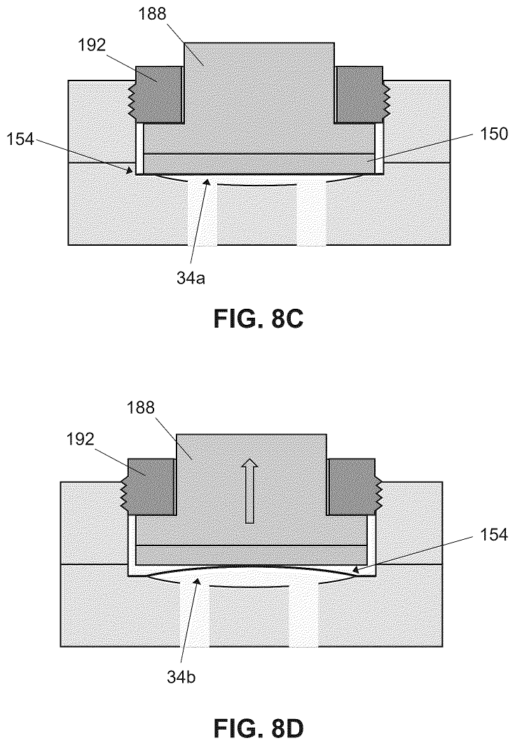

Referring to FIGS. 6D and 6E, accumulator 106 can include a set screw 186 that interacts with flexure arm 146 (e.g., at or near second portion 166). Set screw 186 can be configured to adjust a total volume within chamber 34. For example, set screw 186 can be adjusted to calibrate a displacement amount for each cycle and can be adjusted as necessary to create a threshold (e.g., baseline) volume in chamber 34. In this way and others, an operating volume of chamber 34 can be adjusted or calibrated to ensure accurate dosage of fluid during operation. The calibration of accumulator 106 is discussed in more detail with respect to FIGS. 8A-8D below.

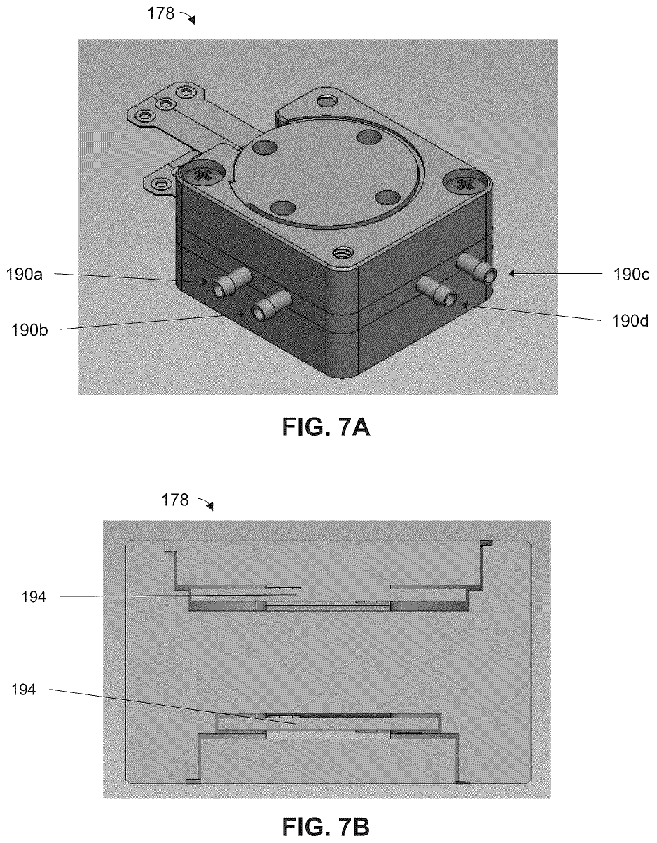

Referring now to FIGS. 7A and 7B, perspective and sectional views of flow control unit (FCU) 178 are depicted, respectively. Flow control unit (FCU) 178 can include or correspond to first and second valves (130 and 134, respectively). FCU 178 can additionally include or define portions of the first, second, third, and fourth channels (e.g., 114, 118, 122, and 126) or connect the channels to their respective components (e.g., connections between channels and valves). For example, FCU 178 can define port(s) 190a, 190b, 190c, and 190d, which can connect to first, second, third, and fourth channels (114, 118, 122, and 126, respectively), to allow for a fluid flow through the FCU. For example, first channel 114 can connect to port 190a to allow for fluid communication between reservoir 30 and FCU 178. Second channel 118 can connect to port 190b to allow for fluid communication between FCU 178 and chamber 34. In a similar manner, third channel 122 can connect to port 190c (to allow for fluid communication between chamber 34 and FCU 178), and fourth channel 126 can connect to port 190d (to allow for fluid communication between FCU 178 and an outlet of IMD 14 such as catheter port 110). First and second valves (130 and 134, respectively) housed within FCU 178 can then selectively allow or restrict the flow of a fluid through the FCU, acting to allow fluid communication between components (e.g., reservoir 30, chamber 34, and catheter port 110) as desired.

As previously described, first and second valves (130 and 134, respectively) can each be actuated between open and closed states (e.g., via control signal(s) 78 from a controller 42). For example, first valve 130 and second valve 134 can include a piezoelectric element 194 that may be actuated to cause the valve to move between the open and closed states. For example, piezoelectric element(s) 194 can be deformed (e.g., via selected application of electricity) to allow passage of fluid through the valve. In some configurations, piezoelectric element(s) 194 can correspond to a disc transducer, stack transducer, bender transducer or the like.

Referring now to FIGS. 8A-8D, calibration of an example of accumulator 106 is illustrated. As shown in FIG. 8A, accumulator 106 may include actuator 38, flexure arm 146, elastomer 150, diaphragm or membrane 154. The second end of flexure arm 146 can include set screw 186 that is configured to contact a cap 188 that is operatively coupled to elastomer 150 such that movement of the cap moves the elastomer. Cap 188 can be coupled to a body of accumulator 106 via a retaining ring 192 that is engaged with the body (e.g., cover, manifold, or both) of the accumulator. Cap 188 and retaining ring 192 can be disposed in a recess formed within the body of accumulator 106 that at least partially defines chamber 34. In the depicted configuration, retaining ring 192 includes an external thread that mates with a thread of accumulator 106 and a position of the retaining ring can be adjusted relative to a body of accumulator 106 by rotating (e.g., tightening or loosening) the retaining ring. As seen in FIG. 8B, cap 188 can abut elastomer 150 such that movement of the cap (e.g., via displacement of set screw 186) can move elastomer 150 and displace diaphragm 154 to dispense fluid from chamber 34.

Set screw 186 and retaining ring 192 can be adjusted to calibrate an operating volume of chamber 34. To illustrate, FIG. 8C shows retaining ring 192 disposed at a first position such that chamber 34 defines a first volume 34a and FIG. 8D shows the retaining ring disposed at a second position such that the chamber defines a second volume 34b that is greater than the first volume. Retaining ring 192 can act as a backstop for diaphragm 154 (e.g., in conjunction with cap 188 and elastomer 150) and the volume of the chamber can be adjusted by tightening or loosening the retaining ring. As shown, diaphragm 154 can be biased in a direction toward cap 188 and retaining ring 192. In some configurations, diaphragm 154 can contact or abut the cap or elastomer 150. In a non-limiting example, tightening retaining ring 192 lowers it (along with cap 188 and elastomer 150) and decreases the overall accumulator volume (e.g., FIG. 8C) while loosening the retaining ring raises the retaining ring relative to the accumulator body and increases the volume (e.g., FIG. 8D). In some configurations, once the volume is set as desired, set screw 186 can be adjusted (e.g., lowered) so that it just contacts cap 188 to facilitate the motion from actuator 38 and flexure arm 146 to be transferred to diaphragm 154. After calibration, retaining ring 192, set screw 186, or both, can be permanently fixed to the accumulator body (e.g., via spot welds or the like) to prevent volume changes during use of IMD 14. Alternatively, IMD 14 can include an actuator (e.g., servomotor) that is configured to interact with retaining ring 192, set screw 186, or both. In some such configurations, the operating volume of chamber 34 can be adjusted (e.g., to a different volume) or calibrated (e.g., changed back to the set volume after errant changes) via actuation of an actuator while IMD 14 remains within the patient.

Some of the present methods can include a manor of calibrating the accumulator. The process may include the steps of opening first valve 130 to fill chamber 34 and closing the first valve. Opening second valve 134 to empty chamber 34 and then closing the second valve. In some configurations, the liquid may be delivered to a capillary or other liquid measuring tube. The opening and closing of the first and second valves can be repeated to flush air from the accumulator and until a liquid level is visible in the capillary. The initial volume of the liquid can then be measured and one or more dispensing cycles may be repeated and the change in volume noted. This volume change can then be utilized to determine the volume of chamber 34. In some configurations, the dispensing cycle includes: opening first valve 130 to fill chamber 34, closing the first valve, opening second valve 134, actuating the actuator 38 (manually or via piezo elements), closing the second valve, and then releasing the actuator. This cycle can be repeated for as many data points as necessary. In this way and others, accumulator 106 can be accurately calibrated to deliver a specific volume of fluid per cycle.

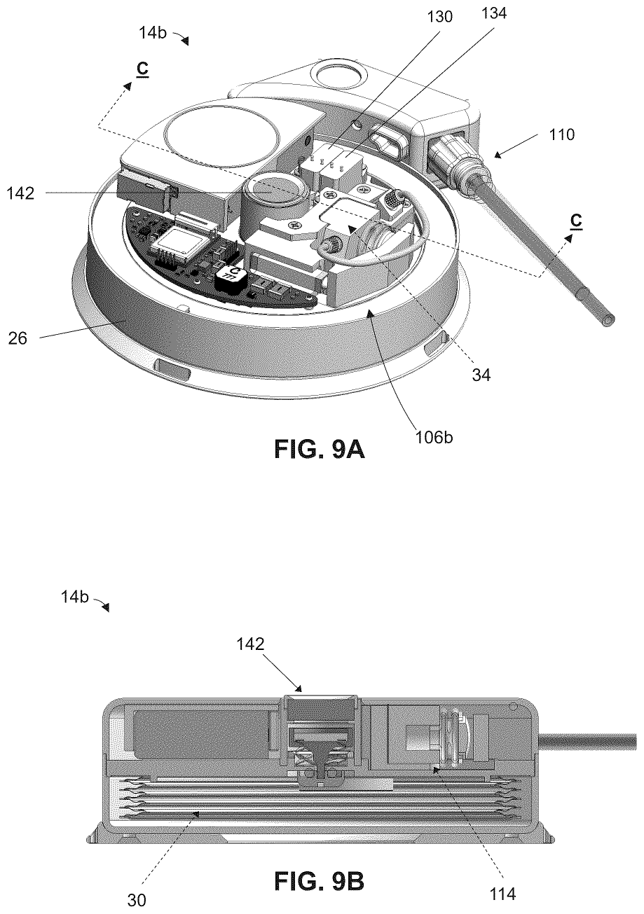

Referring now to FIGS. 9A and 9B, another example of an implantable device, IMD 14b, is depicted with an accumulator 106b. As described herein, accumulator 106b may function in substantially the same manner as accumulator 106. As shown, accumulator 106b can be disposed on housing 26 and can define a chamber 34 that is in fluid communication with reservoir 30 via first channel 114. As described above, accumulator 106b can be configured to pressurize chamber 34 to force fluid to catheter port 110 via actuation of second valve 134. The depicted reservoir 30 can operate in the same manner as described above and may be pressurized via a liquid surrounding the reservoir, a spring, or the like).

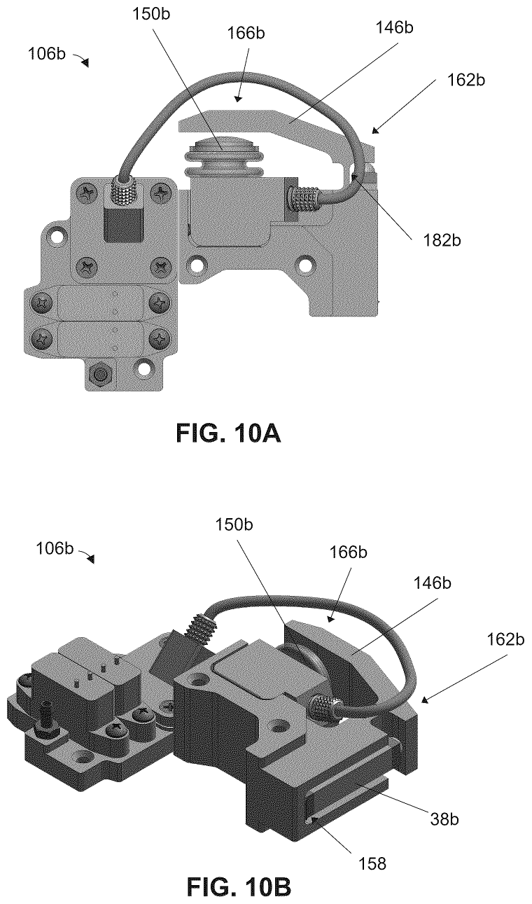

Referring now to FIGS. 10A and 10B, accumulator 106b is shown in greater detail. Accumulator 106b can include an actuator 38b, flexure arm 146b, bellows 150b, or combination thereof. In some configurations, accumulator 106b can include other components that are not depicted, such as a diaphragm or other components described herein. As is depicted, flexure arm 146b is employed, which can be actuated by piezoelectric element(s) 158 to rotate around pivot point 182b, to exert a force on bellows 150b. In some configurations, bellows 150b can be a hydraulic bellows that is filled with a fluid, such as a non-compressible fluid.

Similar to accumulator 106, an electric current can be supplied to expand the piezoelectric element(s) 158 in a first direction to exert a force on a first portion 162b of flexure arm 146b. Acting around pivot point 182b, the force on first portion 162b of flexure arm 146b can cause a rotational motion, where a second portion 166b of the flexure arm can then exert a force on bellows 150b in a second direction, where the force on the elastomer can act to increase the pressure inside of the chamber (e.g., 34b). The first and second directions D1 and D2 can be orthogonal such that the first direction force of piezoelectric element(s) 158 can be transformed (e.g., by means of pivot point 182b and flexure arm 146b) into the second direction force which can act on bellows 150b, thereby expanding diaphragm 154b to constrict the chamber (e.g., 34b) and increase the pressure therein. The chamber (e.g., 34b) housed within accumulator 106b can be utilized in a similar manner to the chamber 34 of accumulator 106, where a fluid can be deposited within the chamber, the pressure of the chamber can be increased, and the fluid can ultimately be delivered to a patient (e.g., 50).

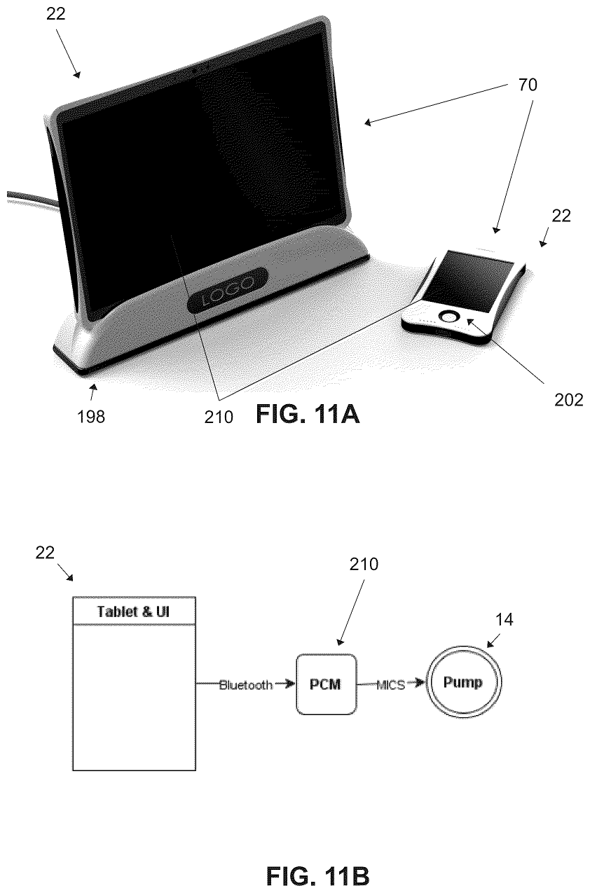

Referring now to FIGS. 11A and 11B, various examples of electronic accessories 22 are depicted. As shown in FIG. 11A, electronic accessories 22 can include a professional device 198 and a user device 202. Electronic accessories 22 can be in communication with IMD 14 and can be configured to provide one or more indications to a user (e.g., notifications, alerts, data, or other information). For example, electronic accessories 22 can include one or more input/output (I/O) devices 70 that can include or correspond to displays, touchscreens, speakers, light sources, vibrators, buttons, keys (hard or soft), microphone, or other types of components that enable a user to receive information from, or provide information to, the electronic accessories. Although not shown, professional device 198 and user device 202 can include components (e.g., hardware and software) associated with conventional mobile computers such as smart phones, tablets, laptops, or the like. For example, professional device 198 and user device 202 can include one or more controllers, processors, memories, transceivers, sensors, actuators, interfaces, or other the like. To further illustrate, in a non-limiting example, external device(s) (198 or 202) can include a pressure sensor configured to determine ambient pressure. The ambient pressure can be sent to IMD 14, which can adjust one or more operations. As an example, a measurement associated with the ICP waveform can be adjusted based on the ambient pressure to provide a gauge pressure measurement. Additionally, or alternatively, one or more operations can be adjusted based on an ambient pressure measurement being outside of a threshold. Because external devices can be carried by a patient, the pressure readings (e.g., absolute pressure measurements) of external devices (198, 202) can be at the same ambient pressure as IMD 14 and can provide accurate pressure readings if a patient experiences large elevation changes, such as when travelling.

Electronic accessories 22 can be configured to perform one or more operations associated with the operations of IMD 14. For example, electronic accessories 22 can be configured to actuate I/O devices 70 (e.g., between states) based on receiving information from IMD 14, such as information associated with a patient's ICP waveform. For example, user device 202 can be configured to determine an orientation of a patient (e.g., 50) and provide a notification or alert based on the orientation. For example, user device 202 can determine the orientation of a patient based on the orientation of IMD 14 and, based on the orientation being outside of an inclination threshold, provide a notification (e.g., display a notification on a user interface) to adjust the orientation. To further illustrate, user device 202 can determine a patient (e.g., 50) is upright and send an alert to instruct the patient to lie down to facilitate determination of intracranial pressure or delivery of a pharmaceutical agent. Although IMD 14 is designed to function semi-autonomously (e.g., a medical professional will set the program and fill the reservoir (e.g., 30) with a pharmaceutical agent), user device 202 can provide a degree of control to a patient (e.g., 50) while simultaneously giving feedback and providing data on the patient's status. Professional device 198 can perform the same or similar operations as user device 202 in addition to being able to provide medical information or assist in performing medical operations. Professional device 198 is configured to be used solely by licensed practitioners and can perform operations (e.g., adjusting dosage) that should not be performed by the patient. In some such configurations, professional device 198 can provide instantaneous and adaptive control of IMD 14 during certain scenarios. In this way and others, a doctor can access a variety of data sets 102 and apply thresholds 98, instructions 94, and modify the application(s) 90 in use with system 10 via professional device 198. In a similar manner, professional device 198 and user device 202 can additionally provide alerts to a medical professional or patient (e.g., 50), respectively, of dangerous vital signs, (e.g., ICP being greater than or less than a threshold).

As is depicted in FIG. 11B, system 10 may include an intermediary device 210 (e.g., pump communication module (PCM)). In some such configurations, intermediary device 210 can facilitate communication between professional device 198 or user device 202 and IMD 14. For example, the external device(s) (198 and 202) can communicate with the intermediary device (e.g., PCM 210) via a first protocol and, after the PCM has received communication and/or data, the PCM can communicate with IMD 14 via a second protocol. In some configurations, the first protocol of communication may include Bluetooth or Wi-Fi, and the second protocol of communication can include Medical Implant Communication Service (MICS). MICS communication is a low-power, unlicensed radio service that is widely used to support implanted medical devices. In this way and others, IMD 14 can utilize MICS communication and still communicate with electronic devices that do not support MICS communication. In at least this way, both the medical professional and patient 50 can maintain varying degrees of control over system 10 and tailor system settings in order to provide optimal medical care.

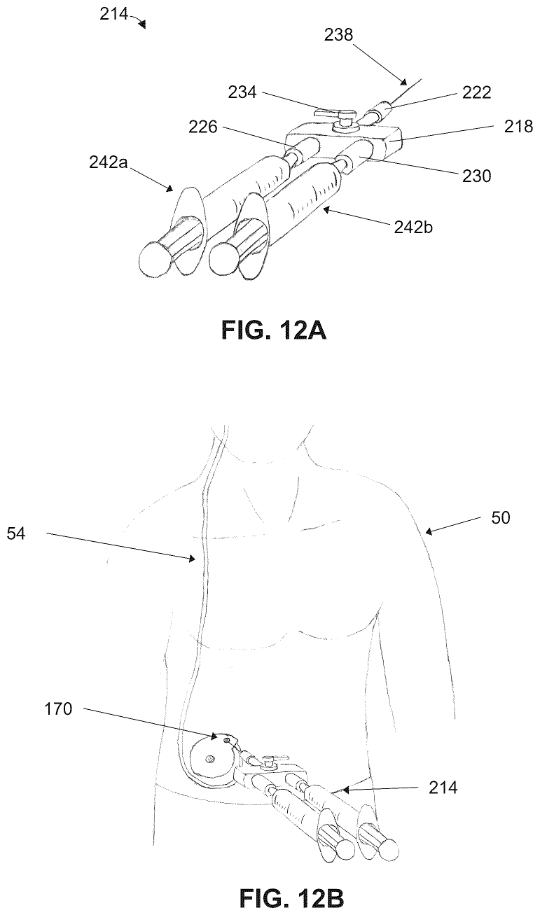

Referring now to FIGS. 12A and 12B, an example of a medical accessory 18 is shown as a multi-chamber syringe 214. Syringe 214 can include a multi-chamber valve 218 having a plurality of ports, such as a first port 222, a second port 226, and a third port 230. Syringe 214 can include a lever 234 or switch that is configured to divert fluid flow through valve 218 between different states. For example, first port 222 can be in fluid communication with second port 226 in a first state and the first port can be in fluid communication with third port 230 in a second state. As depicted in FIG. 12A, a needle 238 can be coupled to first port 222 and plunger type syringes 242a, 242b can be coupled to second and third ports 226, 230, respectively.

Multi-chamber syringe 214 can be utilized to take a sample of or remove cerebrospinal fluid (CSF) from a patient 50. Typically, obtaining a sample of cerebrospinal fluid is difficult and conventional methods require invasive procedures which can cause complications to a patient. Referring to FIG. 12B, needle 238 can be inserted into second access port 170 associated with catheter port 110 such that the needle is in fluid communication with a lumen of catheter 54 implanted in a patient 50. In operation, the lumen of catheter 54 may be filled with a pharmaceutical agent that is being delivered to the treatment area. When syringe 214 is in communication with catheter 54, first syringe 242a (defining a first chamber) can be used to withdraw liquid (e.g., pharmaceutical agent) from the catheter. The volume of the first chamber of first syringe 242a can be greater than or equal to a measured volume of the lumen of catheter 54, thereby allowing all fluid within the catheter to be withdrawn into the first chamber. A second syringe 242b (defining a second chamber) can then be used to collect further fluid from the treatment area. To illustrate, when catheter 54 is disposed within a ventricle or cavity within the brain of patient 50, the negative pressure created during withdrawal of fluid by first syringe 242a will draw CSF into the lumen of the catheter. In this way and others, a sample of CSF can be isolated within the second chamber of second syringe 242b after withdrawal of all of the pharmaceutical agent by first syringe 242a. Lever 234 can be used to switch fluid communication within valve 218 (e.g., from needle 238 to either the first chamber or second chamber of first and second syringes 242a and 242b, respectively), allowing for the separation of fluids. Once an adequate sample of cerebrospinal fluid has been collected, valve 218 can be switched back to allow for the pharmaceutical agent to be disposed back to catheter 54 to continue treatment for patient 50. Therefore, system 10 and multi-chamber syringe 214 can provide a safer and less complicated method to obtain a sample of cerebrospinal fluid. In some configurations, first or second syringes 242a, 242b can have indications (e.g., markings, protrusions, or the like) of the volume of an associated catheter 54 so that an operator will know how much fluid is required to be withdrawn before the CSF can be accessed.

The above described systems can be used in methods for treating patients. Some of the present methods for treating a patient (e.g., 50) can comprise implanting a system (e.g., 10) into the patient with distal end (e.g., 62) of a catheter (e.g., 54) disposed within a region of the patient's brain (e.g., ventricle). If the distal end 62 of the catheter 54 is positioned in a treatment area of the brain (based upon information associated with the ICP waveform), an alert can be initiated at an electronic accessory (e.g., 22) to determine the position of the distal end 62 of the catheter 54 without secondary imaging equipment. In at least this way, a medical professional can confirm correct placement of the distal end 62 of the catheter without or with minimal expensive secondary imaging procedures. A medical professional can then determine a first dosage of an active substance (e.g., pharmaceutical agent) that can be configured to be delivered to the target area. The IMD (e.g., 14) can then be actuated to deliver the first dosage to the target area via the catheter with proximal end (e.g., 74) coupled to the IMD 14. An orientation of the IMD 14 can be determined and based upon the orientation being outside of an inclination threshold (e.g., 98), an alert can be initiated for the electronic accessory 22. Once the orientation is determined to be within the inclination threshold 98, the ICP can be determined. The ICP of the patient can be detected via a sensor (e.g., 46) of the IMD 14, and if the ICP is greater than a maximum threshold, the actuator (e.g., 38) can be prevented from initiating the delivery of a second dosage of the active substance. Transmitting information to an external device (198 and/or 202) can include: transmitting information from the IMD to an intermediary via a first protocol and transmitting information from the IMD to a user device via a second protocol.

Should a sample of cerebrospinal fluid (CSF) need to be gathered, system 10 can allow so without extensive and invasive procedures. A multi-chamber syringe (e.g., 214) can be used to extract substantially all of the active substance within the lumen of the catheter 54 via a first plunger tube syringe (e.g., 242a) and second port (e.g., 226) of the syringe, the first chamber having a volume greater than or equal to a volume of the lumen of the catheter. After extracting the active substance, the CSF can be extracted from the lumen of the catheter 54 via a second plunger tube syringe (e.g., 242b) and third port (e.g., 230) of the syringe. The active substance can then be injected back into the lumen of the catheter 54 from the first plunger type syringe 242a of the multi-chamber syringe 214 to continue medical treatment of the patient 50.

In some configurations, the method can include implanting an IMD into the patient with a distal end of a catheter disposed within an area of resection. In some aspects, the method for treating a patient can include delivering an active substance to a treatment area (e.g., ventricle of the brain, area of resection, or the like). In some configurations, the active substance can include carboplatin, temozolomide, topotecan, or combination thereof. In some such methods, the IMD can deliver carboplatin or temozolomide directly into a resected tumor site for treating cancer (e.g., glioblastoma) to increase localized drug concentration at the tumor site while decreasing systemic toxicity. Such methods can improve over conventional treatments where the blood-brain barrier (BBB) limits chemotherapy penetration.

The above specification and examples provide a complete description of the structure and use of illustrative configurations. Although certain configurations have been described above with a certain degree of particularity, or with reference to one or more individual configurations, those skilled in the art could make numerous alterations to the disclosed configurations without departing from the scope of this invention. As such, the various illustrative configurations of the methods and systems are not intended to be limited to the particular forms disclosed. Rather, they include all modifications and alternatives falling within the scope of the claims, and configurations other than the one shown may include some or all of the features of the depicted configurations. For example, elements may be omitted or combined as a unitary structure, connections may be substituted, or both. Further, where appropriate, aspects of any of the examples described above may be combined with aspects of any of the other examples described to form further examples having comparable or different properties or functions and addressing the same or different problems. Similarly, it will be understood that the benefits and advantages described above may relate to one configuration or may relate to several configurations. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims.

The claims are not intended to include, and should not be interpreted to include, means-plus-or step-plus-function limitations, unless such a limitation is explicitly recited in a given claim using the phrase(s) “means for” or “step for,” respectively.

1. A system comprising:
an implantable medical device (IMD) configured to be disposed within a patient, the IMD comprising:
a housing defining a reservoir configured to receive and store an active substance;
a catheter defining a lumen and having:
a proximal end configured to be in fluid communication with the reservoir;
a distal end configured to be disposed within a treatment area of a brain of a patient;
an actuator configured to initiate delivery of the active substance into the lumen to deliver the active substance to the treatment area.

2. The system of claim 1, comprising:
a pressure sensor disposed within the housing and in fluid communication with the catheter, the pressure sensor configured to detect an intracranial pressure (ICP) waveform when the distal end of the catheter is disposed within the treatment area; and
a controller configured to initiate transmission of information associated with the ICP waveform to an external device.

3. The system of claim 2, where the controller is configured to:
compare an intracranial pressure of the patient to a pressure threshold; and
based on the intracranial pressure being greater than a maximum threshold, prevent the actuator from initiating delivery of the active substance.

4. The system of claim 3, where the IMD is configured to:
detect a reoccurring pressure fluctuation at the treatment area; and
adjust a timing of the actuator to deliver the active substance during a first period associated with a low pressure; or
detect a pressure at the treatment area during the first period.

5. The system of claim 2, where the controller is configured to adjust a timing of a delivery cycle to adjust a dosage of the active substance sent to the treatment area.

6. The system of claim 2, where:
the IMD includes:
an accumulator defining a chamber having:
an inlet in fluid communication with the reservoir; and
an outlet in fluid communication with the proximal end of the catheter;
a first piezoelectric valve disposed between the reservoir and the inlet; and
a second piezoelectric valve disposed between the outlet and the proximal end of the catheter; and
the controller is configured to actuate the actuator, the first piezoelectric valve, and the second piezoelectric valve to deliver a dose of active substance to the treatment area.

7. The system of claim 6, where:
the accumulator includes a diaphragm disposed within the chamber and movable to a pressurized state;
actuation of the actuator causes the diaphragm to be in the pressurized state; and
while the diaphragm is in the pressurized state, actuation of the second piezoelectric valve causes a portion of the active substance within the chamber to move through the outlet into the lumen of the catheter.

8. The system of claim 7, where:
the actuator includes a linear piezoelectric actuator that is movable between an unactuated and an actuated state;
the IMD includes:
an elastomer coupled to the diaphragm and movable between a compressed position and an uncompressed position; and
a flexure arm having a first end disposed adjacent to the actuator and a second end adjacent to the elastomer; and
while the actuator is in the actuated state:
the second end of the flexure arm provides a force to the elastomer;
the elastomer is in the compressed position; and
the diaphragm is in the pressurized state.

9. The system of claim 2, where:
the IMD includes an accelerometer configured to determine an orientation of the IMD; and
based on a determination that the IMD is positioned in a first orientation that is associated with the patient being in an upright position, the controller is configured to transmit a message to the external device;
the controller is configured to adjust an intercranial pressure measurement to account for postural changes of a patient as determined by the orientation of the IMD.

10. The system of claim 2, including the external device in communication with the IMD and where:
the external device includes:
a display
a pressure sensor configured to determine an ambient pressure; and
a controller configured to adjust the information associated with the ICP waveform based on the ambient pressure. ; and
based on the external device receiving the information associated with the ICP waveform, the external device is configured to actuate the display to a first user interface state.

11. The system of claim 2, including the external device in communication with the IMD and where:
the external device includes:
an intermediary device configured to communicate with the IMD via a first protocol; and
a user device that is configured to communicate with the intermediary device via a second protocol; and
where the user device has a display that is configured to display an indication of the ICP waveform.

12. The system of claim 2, including a multi-chamber syringe configured to obtain a sample of CSF from the IMD, the syringe having:
a needle configured to be inserted into the catheter to access the lumen;
a first chamber in communication with the needle, where a volume of the first chamber is greater than or equal to a volume of the lumen; and
a second chamber that is distinct from the first chamber.

13. The system of claim 1, further comprising:
a catheter port configured to receive the active substance from the reservoir;
an accumulator defining a chamber in selective fluid communication with the reservoir;
a moment arm coupled to the actuator; and
where:
the actuator includes a piezoelectric driven actuator disposed within the housing and configured to move between a first state and a second state to initiate delivery of the active substance from the chamber to the catheter port
while the actuator is in the second state, a first end of the actuator deflects a first portion of the moment arm such that a second portion of the moment arm exerts a positive force against the active substance disposed within the chamber.

14. The system of claim 13, further comprising a controller configured to initiate movement of the actuator between the first state and the second state and where the controller is configured to control the actuator in an open loop manner.

15. The system of claim 1, wherein the IMD comprises:
a port configured to be connected to the catheter, the port configured to deliver an active substance from a conduit within the IMD to the catheter;
one or more valves in communication with the conduit, each of the one or more valves actuatable between:
an open state in which fluid can flow through the valve; and
a closed state in which fluid is restricted from flowing through the valve; and
a pressure sensor configured to determine a first pressure within the catheter or target area;
wherein:
at least one of the one or more valves is controlled to perform a first operation in which the at least one valve is actuated from the closed state to the open state to the closed state to deliver the active substance to the conduit;
a timing of the first operation is controlled based on the first pressure.

16. The system of claim 15, further comprising a controller in communication with the pressure sensor and the one or more valves, the controller configured to increase the timing of the first operation based on an the first pressure exceeding a pressure threshold.

17. The system of claim 15, further comprising:
a power source coupled to the one or more valves;
a controller in communication with the pressure sensor and the one or more valves, the controller configured to cause the power source to:
deliver power to the at least one of the one or more valves for a first time period when the first pressure is below a pressure threshold; and
deliver power to the at least one of the one or more valves for a second time period when the first pressure is above the pressure threshold;
wherein the second time period is greater than the first time period.

18. The system of claim 1, wherein the IMD includes a spring disposed within the housing and configured to exert a force on the reservoir.

19. A method of treating a patient, the method comprising:
determining a first dosage of an active substance that is configured to be delivered to a region of a brain;
actuating an implantable medical device (IMD) disposed within a patient to deliver the first dosage to the region of the brain via a catheter having a proximal end coupled to the IMD and a distal end disposed within a skull of the patient;
detecting an intracranial pressure of the region of the brain via a sensor of the IMD;
determining whether the intracranial pressure is greater than a pressure threshold; and
based on the intracranial pressure being greater than the pressure threshold, prevent the actuator from initiating delivery of a second dosage of the active substance.

20. The method of claim 19, comprising:
determining a period of low pressure at the distal end of the catheter;
determining a period of high pressure at the distal end of the catheter;
adjusting an operation to occur during the period of low pressure.

21. The method of claim 19, comprising:
detecting an intracranial pressure (ICP) waveform of the brain;
transmitting information associated with the ICP waveform to an external device;
determining an orientation of the IMD;
based on the orientation being outside of an inclination threshold during detection of the ICP waveform, initiating an alert at the external device; and
based on the orientation being within the inclination threshold, determining the intracranial pressure.

22. The method of claim 19, comprising;
detecting an intracranial pressure (ICP) waveform of the brain;
transmitting information associated with the ICP waveform to an external device;
determining the distal end of the catheter is positioned in a treatment area of the brain based on information associated with the ICP waveform; and
based on the determination that the distal end of the catheter is positioned in the treatment area, initiating an alert at the external device;
where the determination of the position of the distal end of the catheter is performed without imaging equipment.

23. A method of operating an implantable medical device (IMD) configured to be disposed within a patient and deliver an active substance to a catheter having a distal end positioned at a target area, the method comprising:
determining a pressure within a treatment area;
calculating an expulsion time to expel substantially all fluid within a chamber of an IMD; and
setting an actuation time of a valve to be substantially equal to the expulsion time;
wherein the actuation time of the valve includes a time that electrical current is actively delivered from a power source to the valve.

24. The method of claim 23, further comprising:
detecting a change in the pressure of the treatment area;
calculating a new expulsion time based on the change in pressure; and
setting the actuation time of the valve to be substantially equal to the new expulsion time.

25. A method of operating an implantable medical device (IMD), the method comprising:
delivering an active substance from a chamber of the IMD to a treatment area of a patient via a catheter in fluid communication with the chamber;
wherein the treatment area includes an area of resection.

26. The method of claim 25, wherein:
the active substance is carboplatin or temozolomide; or
wherein the method is a method of treating glioblastoma.
